(19) United States
(12) Patent Application Publication
(10) Pub. No.: US 2009/0068646 A1
(43) Pub. Date: Mar. 12, 2009

(54) METHODS AND KITS FOR DETECTING MUTATIONS

(75) Inventors: Jeffery Bacher, Madison, WI (US); Richard Halberg, Madison, WI (US); Marijo Kent-First, Starkville, MS (US); Keith V. Wood, Mt. Horeb, WI (US)

Correspondence Address: MICHAEL BEST & FRIEDRICH LLP, ONE SOUTH PINCKNEY STREET, P O BOX 1806, MADISON, WI, 53701, US

(73) Assignee: PROMEGA CORPORATION, Madison, WI (US)

(21) Appl. No.: 11/577,646

(22) PCT Filed: Oct. 24, 2005
(86) PCT No.: PCT/US05/38433

§ 371 Date: Nov. 13, 2007

Related U.S. Application Data
(60) Provisional application No. 60/621,277, filed on Oct. 22, 2004.
(60) Provisional application No. 60/661,646, filed on Mar. 14, 2005.
(60) Provisional application No. 60/697,778, filed on Jul. 8, 2005.

(51) Int. Cl. C12Q 1/68

(52) U.S. Cl. 435/6

(57) ABSTRACT

Disclosed are methods and kits for detecting mutations in DNA by comparing the size of an amplified microsatellite locus to the expected size. The methods and kits may used in various applications, including monitoring exposure of a cell or organism to a mutagen, evaluating the mutagenicity of an agent, and evaluating a putative precancerous or cancerous cell or tumor cell for microsatellite instability.

### CROSS-REFERENCE TO RELATED APPLICATIONS

This application claims priority to U.S. Provisional Application No. 60/621,277, filed Oct. 22, 2004, to U.S. Provisional Application No. 60/661,646, filed Mar. 14, 2005, and to U.S. Provisional Application No. 60/697,778, filed Jul. 8, 2005, each of which is incorporated by reference, and is being filed simultaneously with an application entitled “Methods and Kits for Detecting Germ Cell Genomic Instability”, filed Oct. 24, 2005 under the Patent Cooperation Treaty, which is incorporated by reference.

### STATEMENT REGARDING FEDERALLY SPONSORED RESEARCH

This invention was made with Government support under grant ______ awarded by the NASA. The United States Government has certain rights in the invention.

### INTRODUCTION

Exposure to mutagens in the environment can pose a serious health threat, particularly to workers in certain high risk occupations. Accurate methods for measuring mutations are critical to estimating potential health risks associated with exposure to radiation and other mutagens. Dosimetry systems provide information concerning the extent of exposure, information that is useful in instituting measures to reduce risk of further exposure. Biological dosimetry provides additional information concerning how radiation affects the individual receiving the radiation. Gross chromosomal changes can be detected by fluorescence in-situ hybridization (“FISH”), a biodosimetric method. However, the accuracy of long-term biodosimetry by cytogenetic means is affected by the loss of chromosomal aberrations over time.

Nearly one-third of the human genome is composed of DNA repeats. Repetitive DNA sequences have been identified as susceptible to mutation in response to mutagens. Microsatellite loci are a class of DNA repeats, each of which contains a sequence of 1-9 base pairs (bp) that is tandemly repeated. Loci having larger repeat units of 10 to 60 bp are typically referred to as minisatellites. Microsatellites and minisatellites are inherently unstable and mutate at rates several orders of magnitude higher than non-repetitive DNA sequences. Due to this instability, microsatellites and minisatellites have been evaluated for increased mutation rates after exposure to mutagens.

Ishizaki et al. (Aviat Space Environ Med 2001 72(9):p. 794-8) examined the effect of radiation exposure (0.02 Gy) on mismatch repair deficient colon cancer cells aboard a 9-day space shuttle flight using six microsatellite loci, including the mononucleotide repeat marker BAT-26. No increase in mutation rate was observed relative to controls. In view of the relatively low radiation dose, this result was not unexpected. Similarly low doses of radiation did not cause a significant increase in chromosomal aberrations in astronauts using standard cytogenetic chromosomal analysis.

Boyd et al. (Int J Radiat Biol, 2000. 76(2):2.169-176) reported a dose-response relationship for radiation-induced mutations at mini- and microsatellite loci in human somatic cells. Various sizes of minisatellite loci were analyzed; microsatellite loci analyzed were di- and tetranucleotide repeats. Boyd identified that the microsatellites were less sensitive than the minisatellites. See Boyd, FIG. 2, page 172.

Microsatellite markers were reported to be altered in A-bomb survivors with leukemia. Nakanishi et al. (Int J Radiat Biol, 2001. 77(6):p. 687-94) analyzed leukemia cells from 13 individuals with acute myelocytic leukemia and with a history of radiation exposure, and from 12 individuals with acute myelocytic leukemia and without a known history of exposure using 10 microsatellite markers, including the mononucleotide repeat marker BAT-40. Estimated radiation exposures ranged from 0.05 to over 4 Gy. Microsatellite Instability (MSI) analysis revealed a high frequency of multiple microsatellite changes in the exposed individuals (85%) compared with non-exposed individuals (8%). Those patients exposed to >1 Gy exhibited a high frequency of MSI (MSI-H), with mutations in greater than 30% of markers. However, only 3 of 13 A-bomb survivors exhibited changes in BAT-40, compared with 2 of 12 non-exposed leukemia patients, which suggests that there is no difference in the stability of BAT-40 in exposed or unexposed patients. Therefore, it appeared that BAT-40 was not sensitive enough to allow detection of radiation-induced mutation. The latter finding is consistent with an earlier report by Okuda et al. (J Radiat Res (Tokyo), 1998. 39 (4):p. 279-87) that exposure to 2 Gy X-rays did not result in increased mutations of BAT-26. Therefore, it appeared that BAT-40 and BAT-26 were not sensitive enough to allow detection of radiation-induced mutation.

Accordingly, persons in the art had come to believe that minisatellites were better able to detect radiation-induced mutations. Furthermore, it was expected that this finding applied to any mutation regardless of what mutagen was the cause of the mutation. For example, Dubrova identified minisatellites as the most unstable in the human genome. Swiss Med Wkly, 2003, volume 133 pages 474-478.

Yamada examined the mutation frequency of G17 and A17 mononucleotide repeats and (CA)17 dinucleotide repeat in human cells lines exposed to oxidative stress (Environmental and Molecular Mutagenesis, (2003) 42:75-84). No effect was observed for either mononucleotide locus, and a small increase in mutation frequency was observed for the dinucleotide locus.

A relatively high level of chromosomal alterations occur on the Y chromosome due to the presence of repetitive elements clustered along the length of the chromosome and the inability of the Y chromosome to participate in recombination repair (Kuroda-Kawaguchi et al. Nature 2001 29:279). The Y chromosome has about 60 million base pairs, of which 95% are in non-recombining regions (NRY) that do not undergo recombination due to the haploid nature of the Y chromosome (Tilford et al. Nature 2001 409:943). Radiation exposure of 1.5 Gy or more often results in persistent azoospermia or reduced sperm production, presumably due to deletions encompassing genes necessary for spermatogenesis (Birioukov, et al. Arch Androl 1993 30(2):99-104; Greiner Strahlenschutz Forsch Prax 1985 26:114-121). Germline mutation rates in short tandem repeats on the Y chromosome are similar to those observed on autosomal chromosomes (i.e., about 1.6×10−3) Bodowle, et al. (Forensic Science International 2005 150(1):1-15). Twelve short tandem repeat loci Y chromosome haplotypes: Genetic analysis on populations residing in North America. Forensic Science International).

Susceptibility to ROS-induced DNA damage is in part a function of DNA sequence, due to intrinsic secondary structural differences between DNA molecules. Lower probabilities of irradiation-induced DNA strand breakage at certain DNA sequences may be explained by reduced minor groove width that limits accessibility to the hydroxyl radical produced by ionizing radiation. Certain secondary DNA structures have been shown to be recognized by DNA repair enzymes and this may also contribute to the relative susceptibility of specific DNA sequences to mutations, particularly some types of repetitive DNA sequences. For example, a 5-bp tandem repeat satellite derived from variants of the core 5′-TTCCA-3′ has been shown to be a “hot spot” for radiation-induced single and double strand breaks (Vazquez-Gundin, F. et al. Radiation Research 2004 157:711-720). This vulnerability of specific sequences may relate to chromatin or tertiary DNA structure that could affect access of hydroxyl radicals to the DNA or exclude water molecules from the proximity of DNA, resulting in lower rate of radiation-induced hydroxyl radicals (Ljungman, M. Radiation Research 1991 126:58-64). The mutagenic potential of different DNA sequences may therefore be due to a balance between specific sensitivities of a particular DNA sequence and protection exerted by DNA structure or chromatin organization or the local sequence environment.

There is a continuing need in the art for methods of assessing exposure to mutation-inducing conditions, such as radiation or chemicals that cause mutations.

### SUMMARY OF THE INVENTION

In one aspect, the present invention provides a method for monitoring an organism or cell population for exposure to a mutagen by amplifying a set of at least one microsatellite locus from a DNA sample from the organism or cell population. The set of microsatellite includes the at least one microsatellite from mononucleotide repeat loci having at least 38 repeats, Y chromosome short tandem repeats of 1-6 bp, or A-rich short tandem repeats having repeating units selected from the group consisting of AAAAG, AAAAC, and AAAAT. The size of the amplification product is compared with the expected size of the amplification product. A difference between the size of amplification product and the expected size of the amplification product is indicative of exposure of the organism or cell population exposure to a mutagen.

In another aspect, the invention provides a method for evaluating the mutagenicity of an agent by exposing an organism or cell culture to the agent and then amplifying a set of at least one microsatellite locus from a DNA sample from the organism or cell culture. The set of microsatellite includes the at least one microsatellite from mononucleotide repeat loci having at least 38 repeats, Y chromosome short tandem repeats of 1-6 bp, or A-rich short tandem repeats having repeating units selected from the group consisting of AAAAG, AAAAC, and AAAAT. The size of the amplification product is compared with the expected size of the amplification product. A difference between the size of amplification product and the expected size of the amplification product is indicative of indicative of mutagenicity.

The present invention also provides a method of detecting microsatellite instability in a human putative cancerous or precancerous cell or tumor cell. A set of at least one microsatellite locus including at least one of a mononucleotide repeat locus having at least 41 repeats and a Y chromosome short tandem repeat of 1-6 bp is amplified from a DNA sample from the putative cancerous or precancerous cell or tumor cell. The size of the first amplification product is determined and compared with the expected size of the amplification product. Microsatellite instability is indicated by a difference between the size of first amplification product and the expected size of the amplification product.

In another aspect, the invention provides a method of detecting microsatellite instability in a mouse putative cancerous or precancerous cell or tumor cell. A set of at least one microsatellite locus including at least one of a mononucleotide repeat locus having at least 48 repeats is amplified from a DNA sample from the putative cancerous or precancerous cell or tumor cell. The size of the first amplification product is determined and compared with the expected size of the amplification product. Microsatellite instability is indicated by a difference between the size of first amplification product and the expected size of the amplification product.

The invention further provides a method for detecting a mutation in a microsatellite locus by amplifying at least one microsatellite including at least one mononucleotide repeat locus having at least 41 repeats from DNA sample from a human cell line or individual to form an amplification product. The size of the amplification product is determined and compared to the expected size of the amplification product. A difference in size between the amplification product and its expected size is indicative of a mutation in the microsatellite repeat locus.

The invention also provides a method for detecting a mutation in a microsatellite locus by amplifying at least one microsatellite including at least one mononucleotide repeat locus having at least 48 repeats from DNA sample from a mouse cell line or individual organism to form an amplification product. The size of the amplification product is determined and compared to the expected size of the amplification product. A difference in size between the amplification product and its expected size is indicative of a mutation in the microsatellite repeat locus.

Also provided is a method for distinguishing between a mutation or artifact. The method involves amplifying a mono-, di- tri-, tetra-, penta-, or hexanucleotide repeat locus from a DNA sample using three different primers. The first primer hybridizes to a first sequence and the second primer hybridizes to a second sequence, the first and second sequences flanking or partially overlapping the target DNA sequence. The third primer hybridizes to a third sequence between the first and second sequences. The DNA between the first and second primers is amplified to form a first amplification product and the DNA between the first and third primers is amplified to form a second amplification product. The sizes of the amplification products are determined and compared to the expected sizes. An equivalent size difference in the first and second amplification products relative to their respective expected sizes indicates a mutation.

In another aspect, the present invention provides a construct comprising a polynucleotide encoding a detectable reporter marker linked to repeat sequence having at least 19 repeats such that a deletion of one or more base pairs of the repeat sequence alters the expression of the reporter marker.

### BRIEF DESCRIPTION OF THE DRAWINGS

FIG. 1 shows the sizes of amplification products of mBAT-59 locus from unexposed (top panel) and irradiated (bottom panel) C57BL/6 cells.

FIG. 2 plots mutation frequency as a function of polyA tract length for various mouse extended mononucleotide repeat markers.

FIG. 3 shows the sizes of amplification products of human extended mononucleotide repeat markers from human fibroblasts exposed to radiation.

FIG. 4 shows the sizes of amplification products of A-rich pentanucleotide repeat markers from human fibroblasts exposed to radiation.

FIG. 5 shows the sizes of amplification products of Y-STR markers from human fibroblasts exposed to radiation.

FIG. 6 plots the mutation frequency of normal human fibroblasts exposed to radiation as a function of dose for Y-STRs (top panel) and extended mononucleotide repeat markers (bottom panel).

FIG. 7 shows the sizes of amplification products of mBAT-59 marker of DNA from old paraquat treated mouse tissue, indicative of ROS-induced muations in mBAT-59 marker.

FIG. 8 shows the sizes of amplification products of mBAT-64 marker of DNA from old paraquat treated mouse tissue, indicative of ROS-induced muations in mBAT-64 marker.

FIG. 9 shows the sizes of amplification products of mBAT-67 marker of DNA from old paraquat treated mouse tissue, indicative of ROS-induced mutations in mBAT-67 marker.

FIG. 10 plots the mutation frequency in short mononucleotide markers (light shading) and in long mononucleotide markers (dark shading) in young and old mice treated with paraquat.

FIG. 11 plots the mutation frequency as a function of poly A length of the marker in mice exposed to oxidative stress.

FIG. 12 shows the sizes of amplification products of DYS349, Penta C, and hBAT-59a markers in human fibroblast cells exposed to ROS.

FIG. 13 compares the size of the predominant allele for each of mBat-24 (A), mBat-26 (B), mBat-30 (C), mBat-59 (D), mBat-64 (E), and mBat-67 (F) from normal intestinal epithelium (top panels) and from tumors (bottom panels) from MMR deficient mice.

FIG. 14 plots the mutation size (bp) observed in mismatch repair (MMR)-deficient tumors for mBat-24, 26, 30, 37, 59, 64, and 67 markers as a function of polyA tract length (bp).

FIG. 15 shows the sizes of mBat-66 markers from small pool PCR of DNA from cell lines derived from C3H mice with radiation induced acute myeloid leukemia.

FIG. 16 shows the sizes of amplification products of mBat-66 markers from small pool PCR of DNA from control C3H mice.

FIG. 17 compares the sizes of amplification products of mBat-54 marker using DNA from paired normal and MMR tumor samples.

FIG. 18 compares the sizes of amplification products of mBat-60A marker using DNA from paired normal and MMR tumor samples.

FIG. 19 is a schematic illustration showing amplification of a marker using three primers to give two products.

FIG. 20 is a mock representation of amplification products of three primer amplification of a marker observed when a true mutation is present.

FIG. 21 shows the amplification products using three primer amplification of mBat-26 marker of DNA from mouse embryonic fibroblasts exposed to 0 Gy (A), or 0.5 Gy (B-E), with the results of Panel B being indicative of a true mutation in mBat-26.

### DETAILED DESCRIPTION OF THE INVENTION

The present invention provides methods for detecting mutations by observing allelic length variations in mononucleotide repeat tracts or in certain other short tandem repeats comprising repeating units of 1-6 base pairs that are sensitive to exposure to mutagens, such as radiation or chemical mutagens.

As used herein, “mutagen” refers to a substance or condition that causes a change in DNA including, but not limited to, chemical or biological substances, for example, free radicals, reactive oxygen species (ROS), drugs, chemicals, radiation and the normal aging process. By “exposing” it is meant contacting a cell or organism with a mutagen or treating a cell or organism under conditions that result in interaction of the cell or organism with a mutagen. It should be understood that “exposing” a cell or organism to a mutagen does not necessarily require an active step. Rather, exposure of a cell or organism to a mutagen may result from the cell or organism being present in an environment in which the mutagen occurs.

The methods allow detection and monitoring of genetic damage in individuals exposed to mutagens. Additionally, the methods may be used to measure mutagenesis in response to exposure of cultured cells or experimental animals to mutagens. In one embodiment, the methods may be used to test the mutagenicity of a particular mutagen by exposing a cell or organism to a mutagen or potential mutagen by comparing amplified microsatellite loci of exposed cells to those of a non-exposed cell or organism. In another embodiment, a cell or organism cell or organism carrying a polynucleotide encoding a detectable reporter marker linked to a microsatellite repeat locus having at least 19 repeats such that a deletion in the microsatellite repeat on exposure to a mutagen alter expression of the reporter marker.

As described in the Examples, numerous extended mononucleotide repeats (i.e., mononucleotide repeats containing from 38-200 repeats) in human or mouse DNA were identified in a search of available sequence information (Tables 1A-1D). Extended mononucleotide repeat sequences have not previously been evaluated for use in detecting an increase in instability in response to environmental insults (i.e., mutagens) or to identify conditions associated with mismatch repair deficiency because relatively long repeats were generally thought to be too highly mutable to afford meaningful results. The general suitability of extended mononucleotide repeats for use in monitoring exposure to mutagens was evaluated using select extended mononucleotide repeats, as described in the Examples. The results indicate that mutations in extended mononucleotide repeats occur with higher frequency in cells exposed to mutagens than in control cells. Extended mononucleotide repeat loci, preferably comprise at least 38 nucleotides repeats. Extended mononucleotide repeat loci suitably have repeats of between 38 and 200 nucleotides, between 41 and 200 nucleotides, between 38 and 90 nucleotides, between 41 and 90 nucleotides, between 42 and 90 nucleotides or between 42 and 60 nucleotides.

Similarly, mutations in extended mononucleotide repeats were found to occur with greater frequency in mismatch repair deficient cells than in cells having a functional mismatch repair system. Mononucleotide repeat loci having 41 or more repeats were found to be useful in detected microsatellite instability in mismatch repair in human cells. Extended mononucleotide repeat loci suitably have repeats of between 41 and 200 nucleotides, between 41 and 90 nucleotides, between 42 and 90 nucleotides, or between 42 and 60 nucleotides.

Extended mononucleotide repeat loci are named according to the species, the base contained in the mononucleotide repeat, and the number of times the base is repeated, as reported in deposited GenBank sequences. However, due to variation between individuals and alleles, the number of bases in mononucleotide repeat may be more or fewer than the number indicated in GenBank. For example, mBAT47 is used to designate a mouse sequence with a 47 base adenine repeat with reference to the GenBank sequence. However, different mouse cell lines or individual organisms may contain one or more alleles having fewer than 47 adenine repeats at that locus.

Other loci suitable for use in the methods of the invention include Y chromosome microsatellite loci comprising repeated sequences of from 1-6 bases (YSTRs or YSTR loci). As described below in the Examples, YSTRs exhibit increased mutation rates following exposure to ROS or radiation, relative to that of unexposed cells, and in MMR deficient tumor cells, relative to that of MMR proficient tumor cells. YSTRs suitable for use in evaluating exposure to a mutagen or in evaluating the microsatellite instability of a putative precancerous or cancerous cell or tumor cell include, but are not limited to, DYS438, DYS389-II, DYS390, DYS439, DYS392, DYS385b, DYS19, DYS389-I, DYS385a, DYS393, DYS437, and DYS 391. It is reasonably expected that other YSTR loci of the Y chromosome will be suitable for detecting ROS or radiation exposure, or microsatellite instability of putative precancerous or cancerous cells or tumor cells, including, but are not limited to, Y chromosome microsatellite loci shown in Table 7, which were identified in a search of available sequence information (i.e., DYS453, DYS456, DYS446, DYS455, DYS463, DYS435, DYS458, DYS449, DYS454, DYS434, DYS437, DYS435, DYS439, DYS488, DYS447, DYS436, DYS390, DYS460, DYS461, DYS462, DYS448, DYS452, DYS464a, DYS464b, DYS464c, DYS464d, DYS459a, and DYS459b). It is specifically envisioned that any other mono-, di-, tri-, tetra-, or pentanucleotide repeat on the non-recombining regions (NRY) of the Y chromosome would be suitable in the methods of the invention.

Methods that identify mutations in microsatellite loci may be used to evaluate exposure to mutagens, including those that cause oxidative stress. Mutations in microsatellite loci are generally found in non-coding regions, and are not deleterious to the cell. Thus, mutations in non-coding repetitive sequences can accumulate, providing a stable molecular record of DNA damage from past exposures.

Accumulation of reactive oxygen species (ROS), which occurs with aging or in response to exposure to certain chemicals, results in mitochondrial DNA (mtDNA) deletions and defective repair of DNA damage. Oxidative DNA damage by elevated ROS is characterized by the production of superoxide anions (O2-), hydrogen radicals (OH) and their common product hydrogen peroxide (H2O2). Accumulation of ROS causes damage to macromolecules, including lipid peroxidation, oxidation of amino acid side chains, formation of DNA-protein cross-links, oxidation of polypeptide backbones resulting in protein fragmentation, DNA damage and DNA strand breaks. Mitochondrial DNA is composed of a 16,569 bp closed circular double stranded genome, and exhibits a common 4977 bp deletion (Δ-mtDNA4977) that has been reported to increase with age and mitochondrial degeneration. Mitochondrial DNA is particularly susceptible to damage by ROS. Damage by hydrogen peroxide is more extensive in mtDNA than in nuclear DNA, and the mutation frequency of mtDNA is 10-1000 fold higher than in nuclear DNA.

As described in the Examples, the effect of accumulated ROS due to aging or exposure to paraquat was evaluated in C57BL/6 mice by examining mtDNA deletion (Δ-mtDNA4977) and genomic stability as measured by mutations in mononucleotide repeat loci. Paraquat is an herbicide that reacts with molecular oxygen in vivo to form ROS. Mutations were detected by amplifying DNA samples containing mBat-24, mBat-26, mBat-30, mBat37, mBat-59, mBat-64, or mBat-67. The results indicated that extended mononucleotide repeats are more susceptible to ROS-induced deletion mutations than are shorter mononucleotide repeats, and that amplification of the extended mononucleotide repeats provided a more sensitive test for ROS damage.

Mutational load profiling, through analysis of changes in mononucleotide repeat sequences over time, is a non-invasive and generalized approach for monitoring an individual's cumulative record of mutations. This approach is useful in predicting and minimizing health risks for individuals exposed to mutagen. The methods of the invention can be used measure genetic damage to cell cultures or whole animals caused by exposure to drugs or chemicals.

In addition, detection of mutations in extended mononucleotide repeat will facilitate detection of tumors or other conditions associated with mismatch repair deficiencies. Individuals with hereditary non-polyposis colorectal cancer (HNPCC) carry germline mutations in DNA mismatch repair genes including MLH1 and MSH2. Individuals with these mutations are predisposed to the development of cancer of the colon, as well as other tissues, especially the endometrium in females. Microsatellite loci mutations occur more frequently in colorectal tumors and other mismatch repair (MMR) deficient cancer cells, presumably because the cells are deficient in MMR. Detection of increased microsatellite instability in a tumor cell provides important diagnostic information relevant to treatment and prognosis. As illustrated in the Examples, amplification of mononucleotide repeat loci having 41 or more repeats and YSTRs provides a sensitive and specific means for evaluating microsatellite instability in mismatch repair deficient tumors. In addition, evaluation of extended mononucleotide repeat amplification products is useful in detecting mutations associated with radiation-induced acute myeloid leukemia.

Cells may be considered to be a putative precancerous or cancer cell if, for example, the cells appear atypical microscopically, in culture or are contained in a polyp or other abnormal mass. Microsatellite stability can be assessed by comparing the amplification products from these cells to amplification products from matched normal cells. Normal cells are cells that are microsatellite stable and do not exhibit any precancerous characteristics, including for example, normal blood lymphocytes or normal intestinal cells.

Briefly, methods for monitoring exposure to a mutagen or for evaluating the mutagenicity of an agent involve amplifying a microsatellite locus in a DNA sample using primers that flank or partially overlap the target sequence in an amplification reaction, suitably, a polymerase chain reaction (PCR). Suitably, the microsatellite loci include mononucleotide repeats, preferably mononucleotide repeat loci having at least 38 repeats, Y STRs, and A-rich pentanucleotide repeat loci (i.e., AAAAG, AAAAT, or AAAAC). The upper limit of the size of the target DNA to be amplified will depend on the efficiency of the amplification method. The size of the target DNA may be selected to reduce length variations due to incomplete copying of the target DNA. Preferably, the target DNA is at most about 1000 base pairs in length.

In the Examples, exposure to a mutagen, the mutagenicity of an agent, or microsatellite instability status of a putative precancerous or cancerous cell or tumor cell is evaluated by comparing the size of an amplification products to the expected size of the amplification product. The expected size of the amplification product can be established, for example, using a suitable control cell. For example, a control cell for mutagenicity studies could be cells obtained prior to exposure to an agent, or unexposed cells that are substantially identical to the exposed cells. A suitable control cell for evaluating microsatellite instability may be a normal, non-cancerous, microsatellite stable cell from the same individual. If a microsatellite locus has a predominant allele in the population (i.e., a monomorphic or quasimonomorphic allele), then the expected size of the amplification product could be the size of the predominant allele in the population. Alternatively, the expected size of the amplification product can be established by pedigree analysis.

In the Examples, the sizes of amplified products were evaluated by capillary electrophoresis. However, sizes of amplified products may be assessed by any suitable means, e.g., sequencing alleles, or by observing increased or decreased expression of reporter proteins in cells containing a DNA construct comprising a reporter gene fused to a DNA repeat such that alterations in the length of the DNA repeat result in a frame shift and loss or gain of reporter gene expression, as described in the Examples.

When performing the methods of the invention, the microsatellite loci may be amplified and analyzed individually, or in combination with other loci as part of a panel. Inclusion of multiple loci in a panel increases the sensitivity of the panel. Suitably, at least four different loci are used in a panel when assessing the microsatellite instability of a putative precancerous or cancerous cell or tumor cell. Preferably, at least five loci are evaluated for microsatellite instability. Multiple loci may be amplified separately or, conveniently, may be amplified together with other loci in a multiplex reaction.

In amplifying a repeat locus according to the methods of the invention, one may use any suitable primer pair, including, for example, those described herein below or those available commercially (e.g., PowerPlex®Y System, Promega Corporation, Madison, Wis.). Alternatively, one may design suitable primer pairs that are adjacent to or which partially overlap each end of the locus to be amplified using available sequence information and software for designing oligonucleotide primers, such as Oligo Primer Analysis Software version 6.86 (National Biosciences, Plymouth, Minn.).

Amplification of DNA containing short tandem repeat (STR) loci (i.e., tandem repeats of mono-, di-, tri-, tetra-, penta-, or hexanucleotide sequences) is associated with a high incidence of PCR products that vary in length due to slippage during amplification rather than because of mutations in those loci. This phenomenon, known as stutter artifact, can make it difficult to determine whether a variation in the size of amplification products is due to stutter or a mutation. The present invention also provides a method of amplifying STR loci that facilitates interpretation of results by allowing one to distinguish between artifactual stutter products and allelic variations. The method employs three primer PCR to generate two partially overlapping PCR products of different sizes, each of which contains the STR. If a mutation (i.e., a deletion or addition) occurred in an STR, both PCR products would show a shift in size of the same magnitude. In contrast, it is unlikely that identical stutter would occur in both amplification products. This method is particularly useful in analyzing mutations in a single cell or a small number of cells, or their DNA equivalent (e.g., small pool PCR). The methods may be used in prenatal or preimplantation diagnostic testing.

A reporter system including a microsatellite locus susceptible to mutation on exposure to mutagens will be constructed. The construct will comprise an expression vector comprising a repeat sequence comprising at least 19 repeats mono-, di-, tri-, tetra-, penta-, or hexanucleotide repeats linked to polynucleotide encoding a detectable reporter marker such that a deletion of one or more base pairs of the repeated sequence alters the expression of the reporter marker in a host cell. The system can be used to evaluate the mutagenicity of an agent by contacting the host cell with the agent and detecting a change in expression of the reporter.

A dual reporter system is described as a prophetic example in the Examples below. The dual reporter system described below includes a 5′ sequence encoding firefly luciferase linked to a 3′ sequence encoding Renilla luciferase through a repeat sequence having at least 19 repeats such that the sequence encoding Renilla luciferase is out-of-frame. A functional Renilla luciferase will be not expressed absent a mutation upstream of the Renilla luciferase coding sequence that restores the reading frame. Downstream of, and in-frame with, the Renilla luciferase coding sequence is a sequence encoding a neomycin resistance marker to permit selection of host cells in which expression of neomycin resistance has been restored through an upstream mutation. To reduce background, the repeat sequence is flanked by a 5′ out-of-frame stop codon and a 3′ in-frame stop codon.

Although the Example below describes a construct having dual detectable markers and further including a selectable marker, it is envisioned that a construct according to the invention may suitably include a sequence encoding any reporter linked to a repeat sequence such that a mutation in the repeat sequence alters (increases or decreases) the expression of the reporter. For example, the construct could include a single reporter and a repeat sequence 3′ of the initiation sequence such that a mutation in the repeat sequence alters expression of the reporter.

A reporter may include any polypeptide having a measurable phenotype. Suitable reporters include, but are not limited to, luminescent proteins (e.g., luciferases), fluorescent proteins (e.g., green fluorescent protein), enzymes that catalyze reactions that produce a detectable effect (e.g. β-galactosidase or β-lactamase). For systems employing two reporters, preferably both reporters can be readily quantified in a single sample.

Two different types of reporters can also be combined. For example, β-galactosidase and firefly luciferase could be combined, and both could be detected in a single sample (Dual-Light® Combined Reporter Gene Assay System from Applied Biosystems). Measuring luminogenic and non-luminogenic reporters has been described in US20050164321A1, which is incorporated by reference.

Reporters could be selected such that a second reporter activates or changes the activity of a first reporter (e.g., Fluorescent Resonance Energy Transfer (FRET) or Bioluminescent Resonance Energy Transfer (BRET).

To reduce false positives, a construct may be designed such that sequences encoding two reporter proteins are separated by a viral peptide insert or linker. When a frameshift mutation occurs, the second reporter is expressed as unfused to the first reporter due to a translational effect or “skip” by the ribosomal machinery.

To facilitate the manufacture or cloning of the reporter construct, selectable markers such as antibiotic resistant markers, fluorescent reporters for use in flow cytometry sorting, or an auxotrophic system (Li et al. (2003) Plant 736-747) may be used.

In a dual reporter system, such as that described in the Examples, a fusion between the second reporter (e.g., Renilla luciferase) and a sequence encoding a toxic substance (e.g., Barnase) can be included to select against anything that already includes frameshifts that would otherwise result in false positives.

The following non-limiting examples are intended to be purely illustrative.

### EXAMPLES

### A. Detecting Radiation-Induced Mutations in Cultured Mouse Cells or SupFG1 Mice

Cell culture and irradiation. Immortalized wildtype mouse MC5 embryonic fibroblast cells derived from C57BL/6 mice were grown in standard cell culture conditions. Exponentially growing cells plated in T-25 tissue culture flasks were irradiated at room temperature with a single dose 1 Gy of 1 or 3 GeV/nucleon 56Fe ions accelerated with the Alternating Gradient Synchrotron (AGS) at the Brookhaven National Laboratory at a rate of 0.5 Gy/min. Cells were grown for 3 days post irradiation to allow recovery, trypsinized, concentrated by centrifugation, and frozen at −80 C.

SupFG1 mice (Leach et al. 1996 Mutagenesis 11(1):49-56) were irradiated with 1 or 3 Gy 56Fe high-LET ionizing radiation using the Alternating Gradient Synchrotron (AGS) at the Brookhaven National Laboratory at a rate of 0.5 Gy/min. The mice were maintained for 10 weeks under standard conditions and diet, and then sacrificed. DNA was isolated from blood using standard procedures.

PCR amplification of microsatellite repeats. Genomic DNA from irradiated or control cells was extracted by standard phenol/chloroform extraction methods and quantified by UV spectrometry and PicoGreen dsDNA Quantitative Kit (Molecular Probes, Eugene, Oreg.) following manufacturer's protocol. Mononucleotide repeats with extended poly-A tracts were identified from BLAST searches of GenBank database. Primers for PCR amplification were designed using Oligo Primer Analysis Software version 6.86 (Molecular Biology Insights, Inc., Cascade, Colo.).

Small pool PCR (SP-PCR) amplification of loci containing extended mononucleotide repeats mBat-24, mBat-26, mBat-30, mBat-37, mBat-59, mBat-64, mBat-66, and mBat-67 was performed using fluorescently labeled primer pairs for each loci (Table 2). PCR reactions were performed by using 6-15 pg of total genomic DNA in a 10 μl reaction mixture containing 1 μl Gold ST*R 10× Buffer (Promega, Madison, Wis.), 0.05 μl AmpliTaq gold DNA polymerase (5 units/μl; Perkin Elmer, Wellesley, Mass.) and 0.1-10 μM each primer. PCR was performed on a PE 9600 Thermal Cycler (Applied Biosystems, Foster City, Calif.) using the following cycling conditions: initial denaturation for 11 min at 95° C. followed by 1 cycle of 1 min at 96° C., 10 cycles of 30 sec at 94° C., ramp 68 sec to 58° C., hold for 30 sec, ramp 50 sec to 70° C., hold for 60 sec, 25 cycles of 30 sec at 90° C., ramp 60 sec to 62° C., hold for 30 sec, ramp 50 sec to 70° C., hold for 60 sec, final extension of 30 min at 60° C. and hold at 4° C. The SP-PCR products were separated and detected by capillary electrophoresis using a Applied Biosystems 3100 Genetic Analyzer and data analyzed using AB GeneScan and Genotyper Software Analysis packages to identify presence of microsatellite mutations.

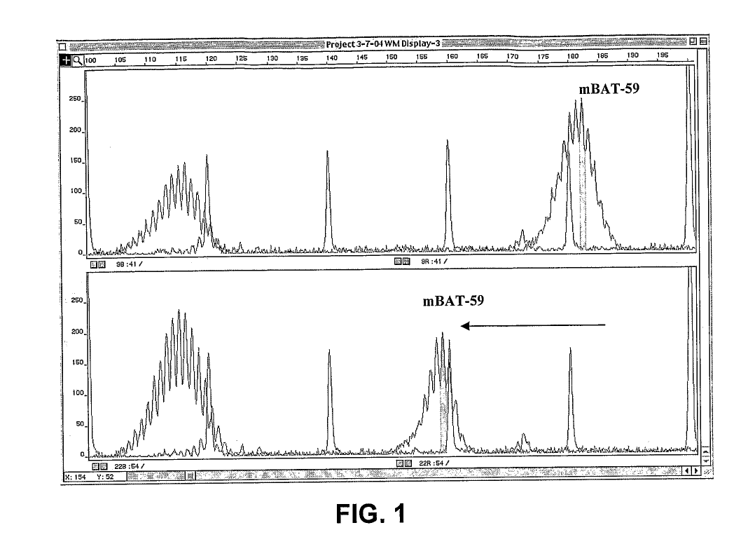

Mutational Analysis. Mutations were not detected in the mBat-24, 26, 30 or 37 markers in DNA isolated from control cells or cells irradiated with 1 Gy iron ions. In contrast, mutant alleles were found with extended mononucleotide repeat marker mBat-59 in 1% (4/408) of alleles from cells irradiated with 1 Gy iron (FIG. 1). No (0/320) mutant mBat-59 alleles were found in control cells. The actual length of the polyA run was estimated to be 51 bp in MC5 cells based on GenBank sequence data. One base pair insertion/deletions mutations were observed in markers with shorter polyA tracts at higher radiation doses, but these also occurred in control cells not exposed to radiation. Therefore, for those markers having shorter polyA tracts, it was not possible to distinguish between true mutations and artifacts generated during the PCR process from repeat slippage or non-templated A addition by the Taq polymerase.

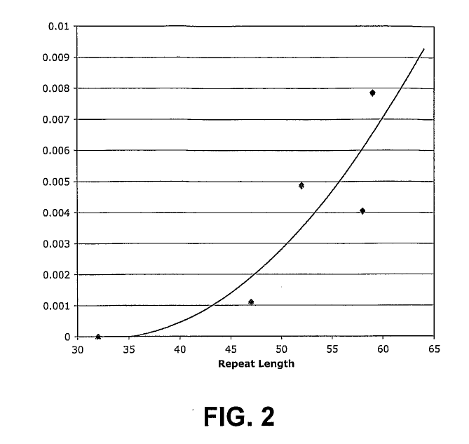

The mutation frequency for mBat-37, 67, 59, 64, and 66 in SupFG1 mice exposed to ionizing radiation was plotted as a function of repeat length (FIG. 2). The predominant repeat length in DNA from unexposed SupFG1 mice for mBat-37, 67, 59, 64, and 66 is 32, 47, 52, 58, and 59 bases, respectively. As can be seen in FIG. 2, the mutation frequency in radiation exposed mice increases as a function of repeat length. In fact, there appears to be an exponential relationship between repeat length and mutation frequency as demonstrated in mouse irradiation experiments.

### B. Detecting Radiation-Induced Mutations in Cultured Human Cells

Cell culture and irradiation. Male human fibroblast cell line #AG01522 from Coriell Cell Repository was grown in DMEM media with 2 mM L-glutamine, 10% fetal bovine serum, 0.5 Units/ml of penicillin, 0.5 μg/ml of streptomycin, and 0.1 mM essential and non-essential amino acids and vitamins (Invitrogen Corporation). Cell cultures were grown at 37° C. and 5% CO2 under sterile conditions. Exponentially growing cells were plated in 25 cm2 tissue culture flasks were irradiated at room temperature with a single dose 0.5, 1 or 3 Gy of 1 GeV/nucleon 56Fe ions accelerated with the Alternating Gradient Synchrotron (AGS) at the Brookhaven National Laboratory at a rate of 0.5 Gy/min. Following irradiation, media was replaced and cells grown for 3 days then collected and frozen at −70° C. until ready for DNA extraction.

Small-pool PCR amplification of microsatellite repeats. Small-pool PCR assays were conducted as described in Example A above using primer pairs (Table 7) to amplify the following microsatellite loci: (1) mononucleotide repeat markers (NR-21, NR-24, BAT-25, BAT-26 and MONO-27); (2) extended mononucleotide repeat markers (hBAT-51d, hBAT-52a, hBAT-53c, hBAT59a, and hBAT-60a); (3) tetranucleotide repeat markers on autosomal chromosomes (D7S3070, D7S3046, D7S1808, D10S1426 and D3S2432); (4) tri-, tetra- and penta-nucleotide repeats on the Y chromosome (DYS391, DYS389 I, DYS389 II, DYS438, DYS437, DYS19, DYS392, DYS393, DYS390, and DYS385); and (5) penta-nucleotide repeats (Penta C and D) (Bacher, J and Schumm, J. Profiles in DNA, 1998 2(2): 3-6; Bacher et al. Disease Markers, 2004 20:237-250.)

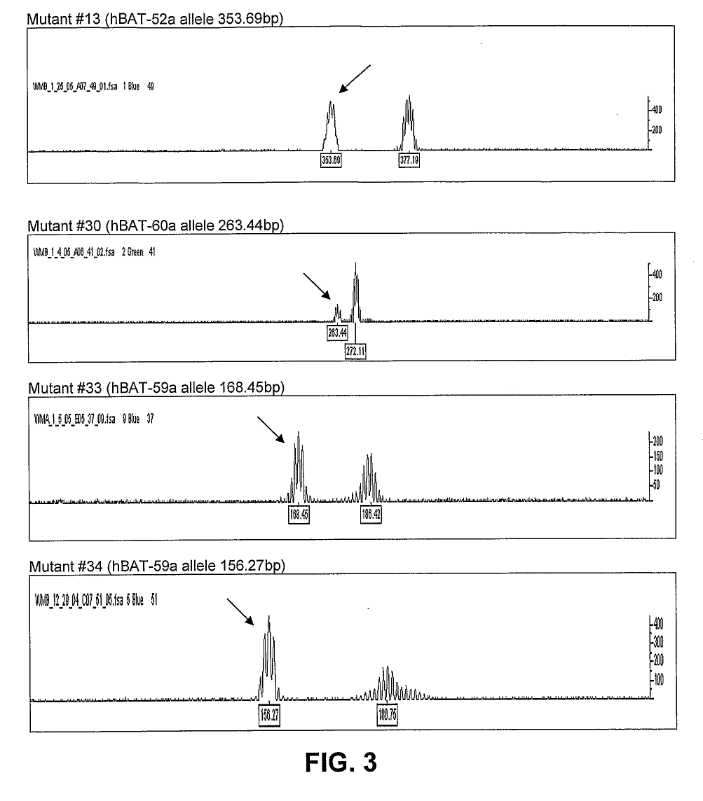

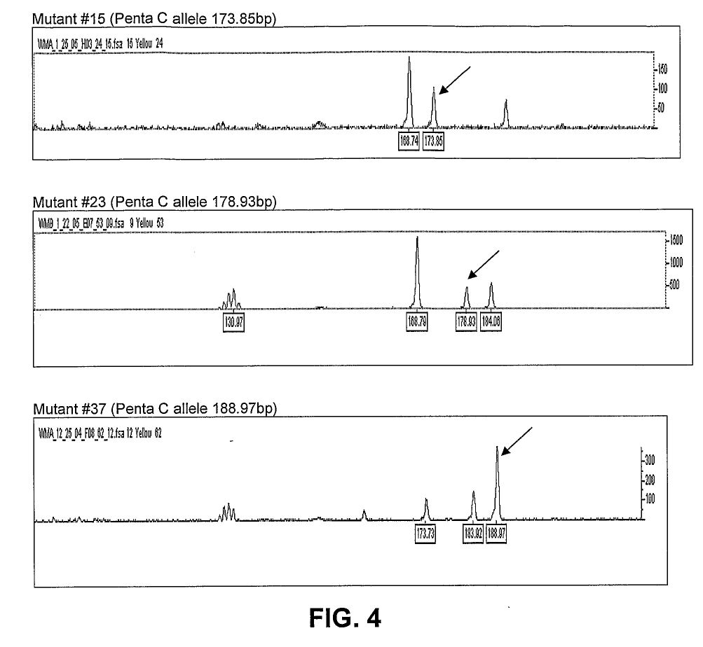

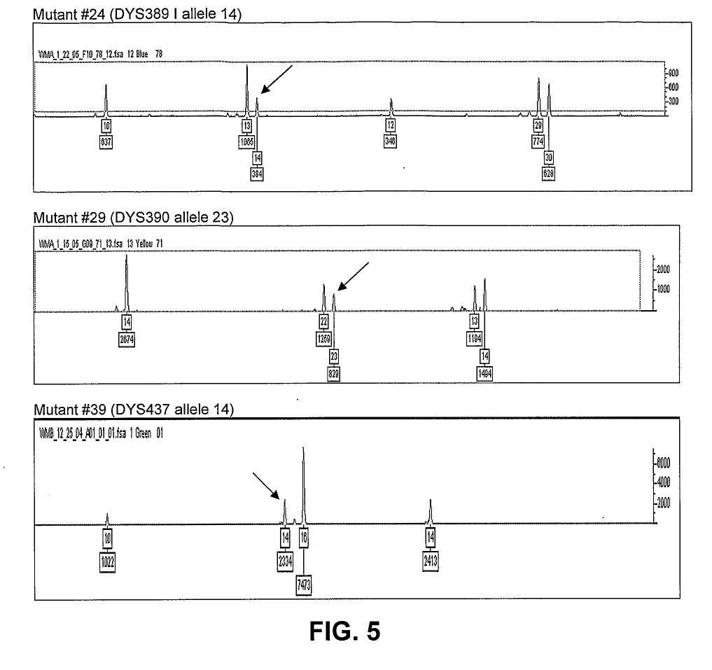

Mutational Analysis. Mutations were detected in the microsatellite repeats of DNA isolated from cells irradiated with 0.5 or 3 Gy iron ions. Mononucleotide repeats with polyA runs of up to 36 bp exhibited little or no increase in mutation rates over controls. Similarly, tetranucleotide repeats on autosomal chromosomes that are sensitive to MSI did not exhibit any evidence of radiation-induced mutations. In contrast, extended mononucleotide repeats with polyA runs of 38 bp or more (FIG. 3) did show statistically significant increase in mutations in irradiated samples as did A-rich pentanucleotide repeats (FIG. 4) and repeats on the Y chromosome (FIG. 5).

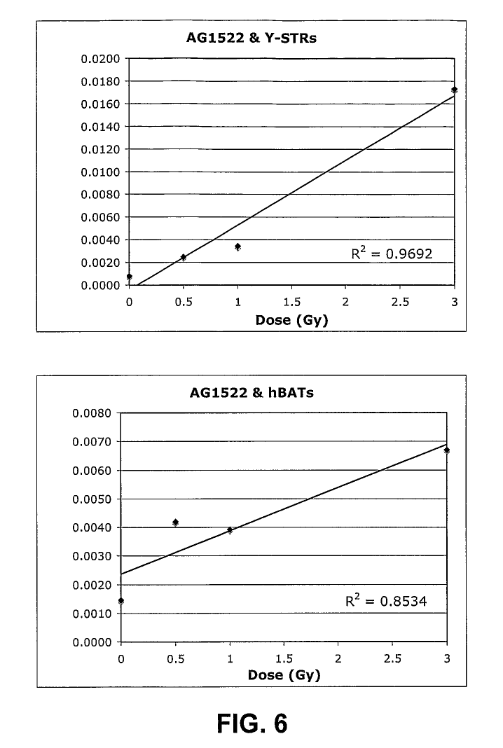

Dose-response curves. A linear dose response was observed for microsatellite markers tested on the Y chromosome and extended mononucleotide repeat markers. Normal human fibroblast cells AG01522 were irradiated with 0, 0.5, 1 or 3 Gy iron ions and the combined mutation frequency of 13 microsatellite markers on the Y chromosome were determined by SP-PCR and plotted (FIG. 6A). There was a good fit to a linear regression line (R2=0.9835), indicating that these markers would be useful for biodosimetry. A linear dose response was also observed for extended mononucleotide repeat markers hBAT-51d, 52a, 53c, 59a, 60a and 62 (FIG. 6B). The observed polyA repeat lengths were estimated based on GeneBank sequence data to be 42, 36, 42, 46, 39 and 36 bp. Mutations were observed primarily in those markers with actual polyA tracts of 38 bp or more.

### C. Detecting Mutations in Mice Exposed to Oxidative Stress

Identification of microsatellite loci and primers. Previously uncharacterized mouse or human mononucleotide repeats were identified through analysis of sequences found in National Center of Biotechnology Information public DNA sequence databases using BLASTN searches for repetitive sequences (Tables 1A-1D). Primers for microsatellite markers were designed with Oligo Primer Analysis software (National Biosciences, Plymouth, Minn.).

Treatment of mice used in paraquat studies. C75BL/6 mice were housed and inbred at the University of Wisconsin, with an average life span 30 months. Four groups of three mice were included in the study: 5-month old mice (young control or YC); 5-month old mice treated with paraquat; 24-month old mice (old age); and 24-month old mice treated with paraquat. Paraquat-treated animals received a single intraperitoneal injection of 50 mg/kg body weight dissolved in PBS 24 hours after their last feeding. Each mouse was sacrificed by cervical dislocation.

Tissue Preparation, DNA Extraction and Quantification. The entire liver from each mouse was dissected, washed with PBS, placed in a 1.5 ml Ependorf tube, snap frozen in liquid nitrogen, and stored at −80° C. DNA was prepared from the mouse liver tissue by using the DNA-IQ Tissue and Hair Extraction Kit (Promega Corporation, Madison, Wis.) and was quantified using the PicoGreen dsDNA Quantitative Kit (Molecular Probes, Eugene, Oreg.) following manufacturers protocols.

mtDNA Deletion Detection. Based on the mouse mtDNA sequence, four fluorescence labeled primer pairs were designed to detect both wildtype sequences and mtDNA deletions. The primer sequences, fluorescent labels, and position are shown in Table 3.

Detection of Mutations in Microsatellites using small pool PCR. Mutations were detected by amplifying loci containing mononucleotide repeats of different lengths using fluorescent labeled primers pairs (Table 2) in multiple replicates of small pool PCR (SP-PCR). The stability of four short mononucleotide repeats (mBAT-24, mBAT-26, mBat-30, mBAT-37) and three extended mononucleotide repeats (mBAT-59, mBAT-64 and mBAT-67) were evaluated.

PCR Conditions. For mtDNA deletion detection, PCR amplification was performed by using 1 ng of total genomic DNA in a 10 μl reaction mixture containing 1 μl Gold ST*R 10× Buffer (Promega, Madison, Wis.), 0.05 μl AmpliTaq gold DNA polymerase (5 units/μl; Perkin Elmer, Wellesley, Mass.) and 0.5 μM mixed primers. PCR was performed on a PE 9600 Thermal Cycler (Applied Biosystems, Foster City, Calif.) using the following cycling conditions: initial denaturation for 11 min at 95° C. followed by 1 cycle of 1 min at 96° C., 10 cycles of 30 sec at 94° C., ramp 68 sec to 62° C., hold for 30 sec, hold for 30 sec, ramp 50 sec to 70° C., hold for 60 sec, 25 cycles of 30 sec at 90° C., ramp 60 sec to 62° C., hold for 30 sec, ramp 50 sec to 70° C., hold for 60 sec, final extension of 30 min at 60° C. and hold at 4° C.

SP-PCR was performed for mutation analysis using 1-2 copies of genomic DNA (6-12 pg). PCR cycles and conditions are the same as described above except that the annealing temperature was 58° C.

Identification of PCR Products. Separation and detection of amplified fragments was performed on an ABI PRISM® 3100 Genetic Analyzer following the manufacturer's protocol (Applied Biosystems, Foster City, Calif.). Data was analyzed with the GeneScan and Genotyper computer software packages (Applied Biosystems).

Statistical Analysis. Statistical analysis was done using the statistical Package Sigma Stat version 3, where P value was determined by using one-way ANOVA for each specific group, using Holm-Sidak method did comparisons between the groups.

Δ-mtDNA4977 detected in old age or paraquat treated mice. Wild type mtDNA is detected by amplification used primers 1, 3, and 4 to yield fragments of 465 bp, 130 bp and 98 bp, respectively. Primer 2 was designed to amplify deleted mtDNA fragments, resulting in a PCR product of 620 bp. No deletion was detected in any of three mice in the young control group (5-month-old mice), whereas one of the three mice in the old age non-paraquat-treated group (25-month-old) showed Δ-mtDNA4977. All three mice in the old age paraquat-treated group showed Δ-mtDNA4977 (Table 4).

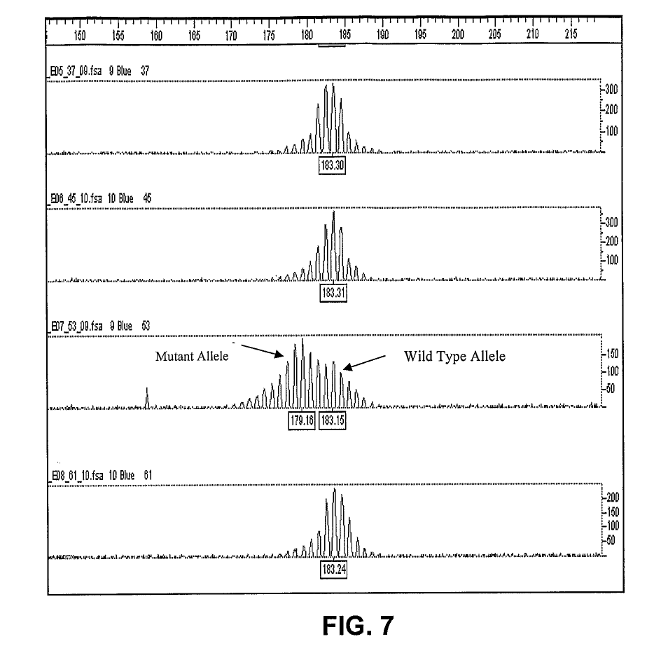

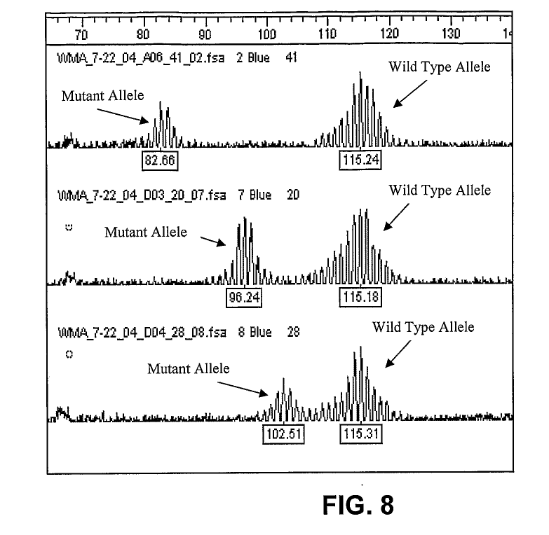

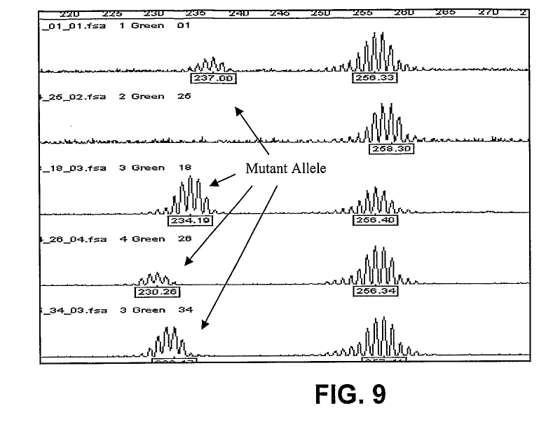

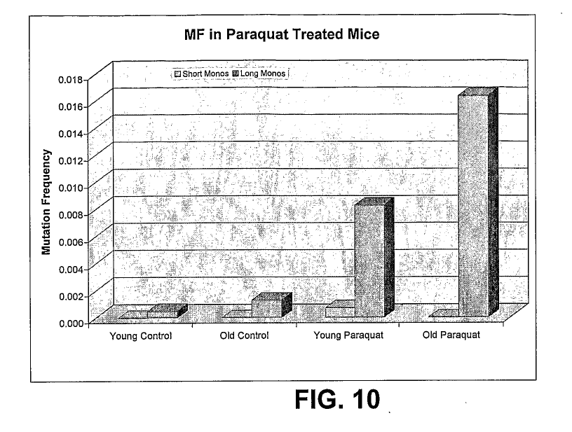

Detection of Mutations by PCR. Analysis of amplification products obtained by PCR amplification of loci mBAT-24, mBAT-26, mBat-30, or mBAT-37 detected no mutations out of 1608 alleles in old paraquat-treated group (Table 5). In contrast, analysis of amplification products obtained by SP-PCR amplification of loci mBAT-59, mBAT-64, mBAT-66 or mBAT-67 showed that the paraquat-treated old age group exhibited mutations in over 1.8% (25/1649) of mononucleotide repeat alleles assayed (Table 5, FIGS. 7, 8, 9, and 10). In mice of the young control group, only one mutant allele out of 2170 alleles was found in any of the extended mononucleotide repeat markers. In the old age control group, analysis of amplification products obtained from SP-PCR replicates identified mutations 3 out of 2342 alleles. The differences in the mutation frequency mean values among the control groups and paraquat-treated groups were statistically significant (P<0.05).

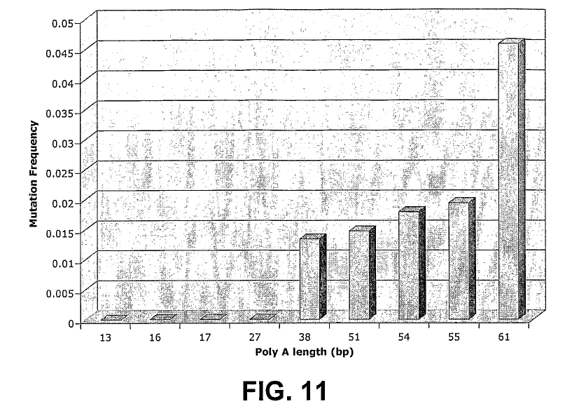

The use of multiple SP-PCR replicates allows detection of mutant alleles that occur less frequently than wild type alleles. The results indicate that extended mononucleotide repeats are more susceptible to mutations in response to oxidative stress than are shorter mononucleotide repeats. Mice exposed to oxidative stress exhibited mutations only in mononucleotide repeats with polyA tracts of 38 bp or more (FIG. 11) Amplification of loci containing extended repeats of 38 bp or greater provides a more sensitive means of detecting ROS-induced mutations.

### D. Detecting Mutations in Human Cultured Cells Exposed to Oxidative Stress

Cell culture. Male human fibroblast cell line #AG01522 from Coriell Cell Repository were cultured in MEM Eagle-Earle BSS 2× concentration of essential and non-essential amino acids and vitamins with 2 mM L-glutamine, 10% fetal bovine serum, 0.5 Units/ml of penicillin, 0.5 μg/ml of streptomycin. Cell cultures were grown at 37° C. and 5% CO2 under sterile conditions and split at a ratio of 1:5 when cells were confluent by releasing cells with trypsin-EDTA treatment. Cells were treated with 0.0 uM (PBS), 0.1 mM, 0.4 mM, 0.8 mM or 1.2 mM hydrogen peroxide diluted in PBS for 1 hour at the same culture conditions described. After treatment, media with hydrogen peroxide was replaced with fresh media and allowed to recover for 3 days. Cells were pelleted and DNA extracted.

Mutation Detection. Mutant alleles were identified by small-pool PCR as described in Example B above using primer pairs specific for microsatellite markers (Tables 2 and 7) including: (1) mononucleotide repeat markers (NR-21, NR-24, BAT-25, BAT-26 and MONO-27); (2) extended mononucleotide repeat markers (hBAT-51d, hBAT-52a, hBAT-53c, hBAT59a, hBAT-60a and hBAT-62); (3) tetranucleotide repeat markers on autosomal chromosomes (D7S3070, D7S3046, D7S1808, D10S1426 and D3S2432); (4) tri-, tetra- and penta-nucleotide repeats on the Y chromosome (DYS391, DYS389 I, DYS389 II, DY8438, DYS437, DYS19, DYS392, DYS393, DYS390, and DYS385); and (5) penta-nucleotide repeats Penta B, C, D, and E (Bacher, et al. 1999. Proceedings from the Ninth International Symposium on Human Identification 1998; and Bacher, et al. Proceedings from the 18th International Congress on Forensic Haemogenetics. 1999).

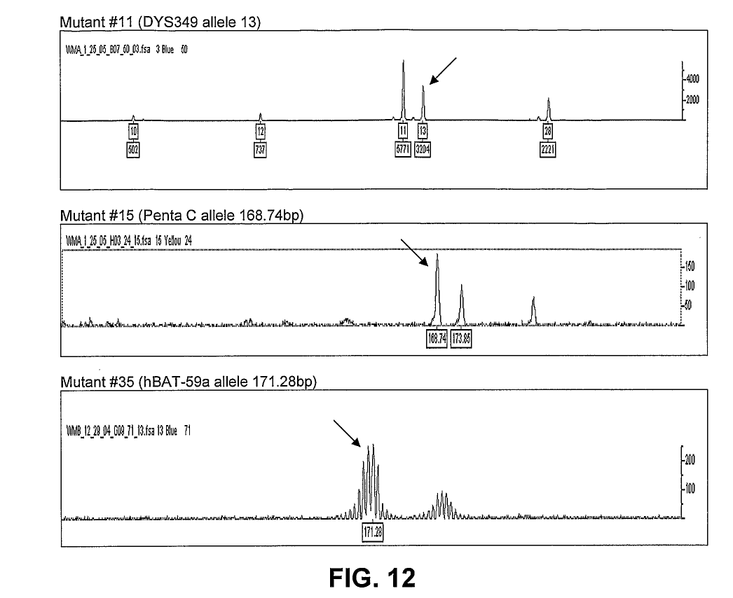

Mutational Analysis of human cultured cells following oxidative stress. Mutations were detected in the extended mononucleotide repeats, Y-STRs and A-rich pentanucleotide repeats in DNA isolated from cells exposed to 0.1 to 1.2 mM hydrogen peroxide (FIG. 12). No mutations (0/1,526 alleles) were observed for short mononucleotide repeat markers NR-21, NR-24, BAT-25, BAT-26 or MONO-27 in cells exposed to hydrogen peroxide.

### E. Detecting Microsatellite Instability in Mouse Tumors

Isolation of DNA from tumor and matching normal tissue samples. C57BL/6 M1h1-deficient mice and B6 Msh2-deficient mice were sacrificed by CO2 asphyxiation. The entire intestinal tract were removed and washed with 1×PBS. Tumors and adjacent normal tissue was removed and snap frozen in liquid nitrogen. DNA was prepared from each sample using DNA IQ chemistry (Promega Corp., Madison, Wis.). In addition, DNA was extracted from leukemia cell lines from C3H mice in which acute myeloid leukemia (AML) had been induced by a whole-body dose of radiation (Pazzaglia et al. 2000 Molecular Carcinogenesis 27(3):219-228).

Detection of microsatellite instability. PCR amplification of loci containing extended mononucleotide repeats mBat-24, mBat-26, mBat-30, mBat-37, mBat-64, mBat-59, or mBat-67 was performed using primer pairs for each loci (Table 2). Amplification of mononucleotide repeats was performed using fluorescently labeled primers in 10 μl PCR reactions containing: 1 μl GoldST*R 10× Buffer (Promega, Madison, Wis.), 0.1-1 μM each primers, 0.05 μl AmpliTaq Gold DNA Polymerase (5 Units/μl; Perkin Elmer, Wellesley, Mass.) per locus and 1-2 ng DNA. PCR was performed on PE 9600 Thermal Cycler (Applied Biosystems, Foster City, Calif.) using the following cycling profile: 1 cycle 95° C. for 11 minutes; 1 cycle 96° C. for 1 minute; 10 cycles 94° C. for 30 seconds, ramp 68 seconds to 58° C., hold for 30 seconds, ramp 50 seconds to 70° C., hold for 60 seconds; 20 cycles at 90° C. for 30 seconds, ramp 60 seconds to 58° C., hold for 30 seconds, ramp 50 seconds to 70° C., hold for 60 seconds; 60° C. for 30 minutes final extension; 4° C. hold. For single template PCR, DNA was diluted to 6-12 pg (1-2 genome equivalents) based on quantification with Picogreen dsDNA Quantitative Kit (Molecular Probes, Eugene, Oreg.) following manufacturer's protocol and confirmed by serial dilution of DNA until PCR failure rates reached 30-50%. PCR amplification was the same as that outlined above except that the number of cycles was increased to a total of 35 cycles.

Separation and detection of amplified fragments was performed on an ABI PRISM® 3100 Genetic Analyzer following the manufacturer's protocol (Applied Biosystems, Foster City, Calif.). Data was analyzed with GeneScan Analysis and Genotyper Software packages from Applied Biosystems to identify predominate allele sizes for each locus. Allelic patterns or genotypes for normal and tumor pairs were compared and scored as MSI-positive if the tumor DNA samples contained one or more alleles not found in normal samples from the same mouse.

The classification of microsatellite instability was based on guidelines suggested by a National Cancer Institute workshop (Boland et al. (1998) Cancer Research 58:5248-5257; Umar et al. (2004) J Natl Cancer Inst 96:261-268, each of which is incorporated by reference herein). Using the Bethesda panel of five microsatellite repeats, tumor samples with 40% MSI were classified as MSI-high (MSI-H), less than 40% as MSI-low (MSI-L), and no alterations were classified as microsatellite-stable (MSS). If more than five markers are to be used, MSI-H group was defined as tumors having MSI at 30% or more of the markers tested, whereas the MSI-L tumors exhibit MSI in 1-29% of the markers.

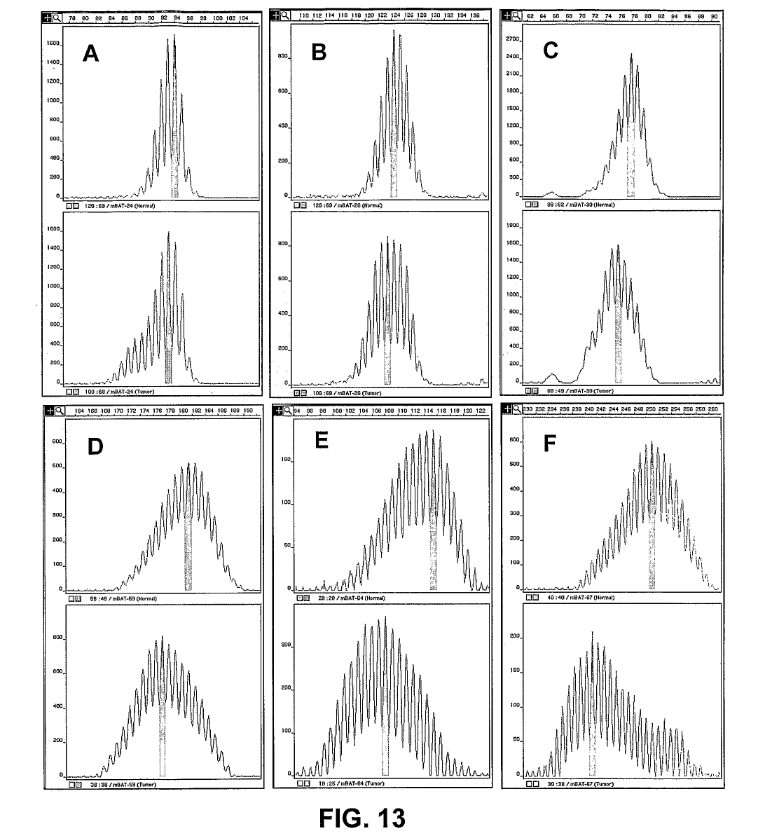

FIG. 13 compares the size of the predominant allele for each of mBat-24 (A), mBat-26 (B), mBat-30 (C), mBat-59 (D), mBat-64 (E), and mBat-67 (F) from normal intestinal epithelium (top panels) and from tumor (bottom panels) from MMR deficient mice. Short deletions of 1-2 bp occurred in mononucleotide repeats with polyA tracts ranging from 24 to 37 (FIG. 13A-C). Much longer deletions (up to 13 bp) were observed in mononucleotide repeats with an extended polyA tract, indicating that larger repeats have larger deletions which are much easier to identify [FIG. 13 D-F]. Mutations in mononucleotide repeats were observed in all 13 tested intestinal tumors lacking mismatch repair activity, with longer repeats having larger deletions (Table 6). The loci having greatest sensitivity as measured by the percentage of MMR deficient tumors exhibiting a mutation in that loci were mBat-26 (85%), mBat-37 (85%), mBat-59 (82%), mBat-64 (72%), and mBat-67 (82%). No changes in allele size were observed in tumors from any of 20 mismatch repair proficient mice tested for MSI using the panel of seven mononucleotide repeats. Taken together, this data demonstrates that extended mononucleotide repeats are highly sensitive to and specific for MSI in mismatch repair deficient tumors. This finding contradicts the accepted hypothesis that longer mononucleotide repeat sequences would be especially susceptible to spontaneous mutations, and would have a spontaneous mutation frequency that was too high, and would thus lack the requisite specificity for MSI analysis. In fact, BAT-40 has been found to lack specificity for detection of MSI-deficient tumors (Bacher et al., (2004) Disease Markers 20: 237-250).

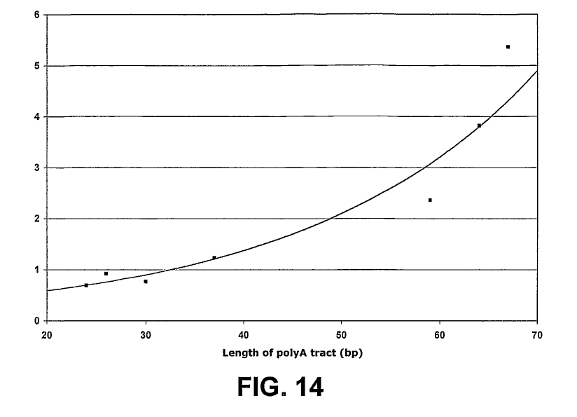

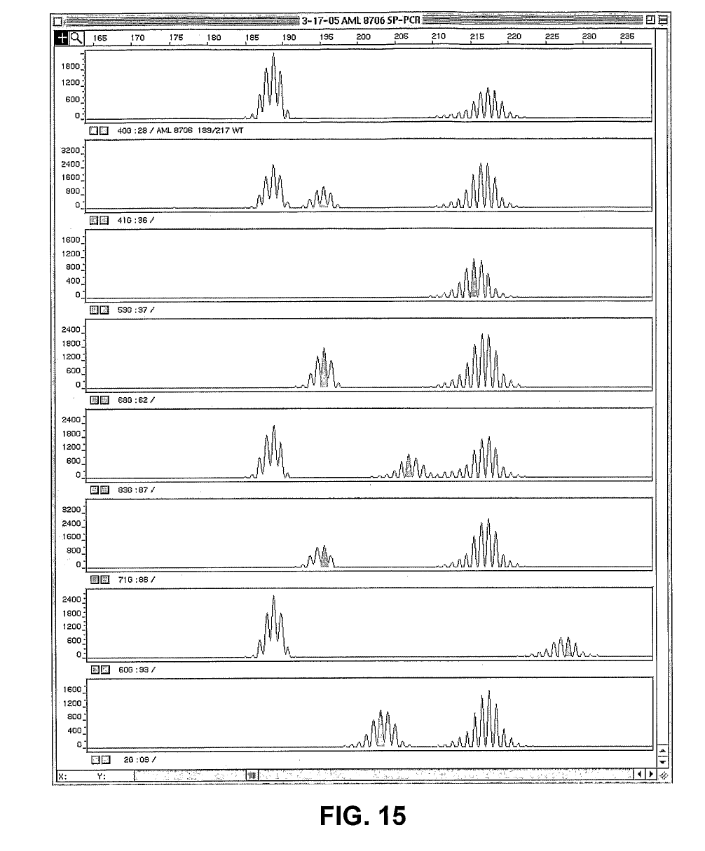

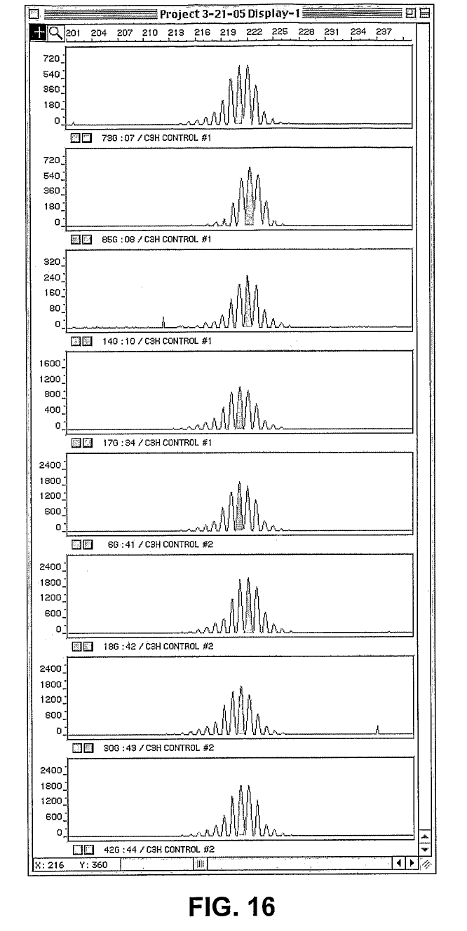

FIG. 14 shows a plot of the size of the mutation (bp) for markers mBat-24, 26, 30, 37, 59, 64, and 67 in MMR deficient mice as a function of polyA tract length (bp). The use of extended mononucleotide repeat markers for MSI detection of mouse tumors overcomes a problem encountered using traditional microsatellite markers, which, typically show only small changes in allele length that are difficult to reliably detect. The minor changes in microsatellite allele length that occurs in mouse tumors probably reflects the short life span of a mouse which limits progressively larger deletions often observed in tumors from other species with longer life spans. Evaluation of MSI in cell lines from C3H mice having radiation-induced acute myeloid leukemia was performed using mononucleotide markers of various lengths. The cells lines exhibited occasional mutations in short mononucleotide repeat tracts (e.g., mBat-30 and mBat-37) with deletions of only 1-2 bp. In contrast, the same cell lines analyzed with extended mononucleotide repeat mBat-66 exhibited a high frequency of variant alleles resembling MSI in mismatch repair deficient tumors (FIG. 15). The mBat-66 was stable in cell lines from C3H mice not exposed to radiation (FIG. 16).

### F. Method of Detecting Microsatellite Instability in Human Tumors

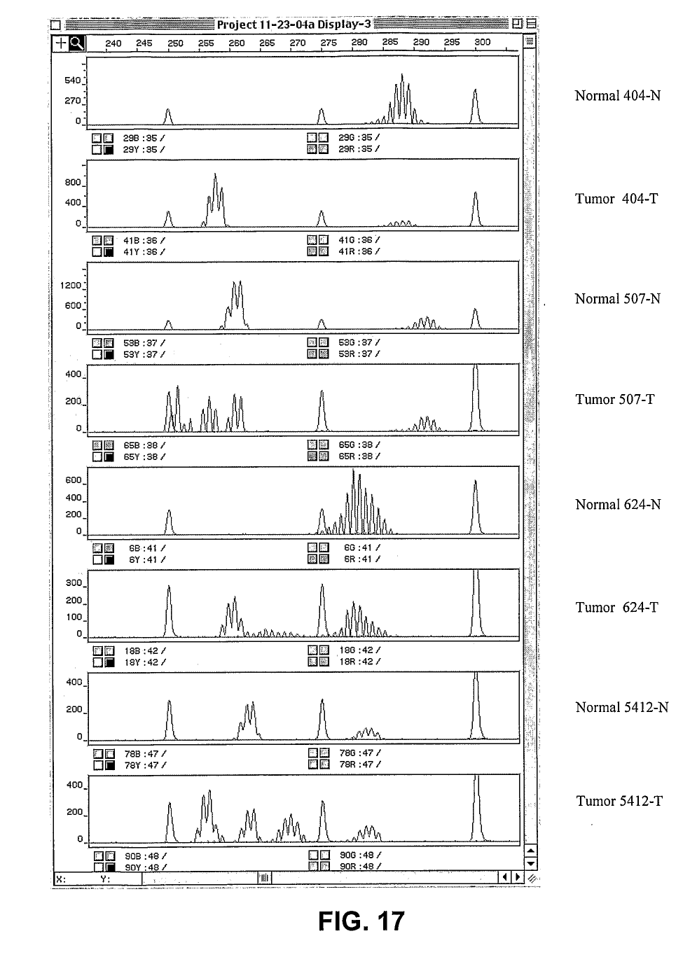

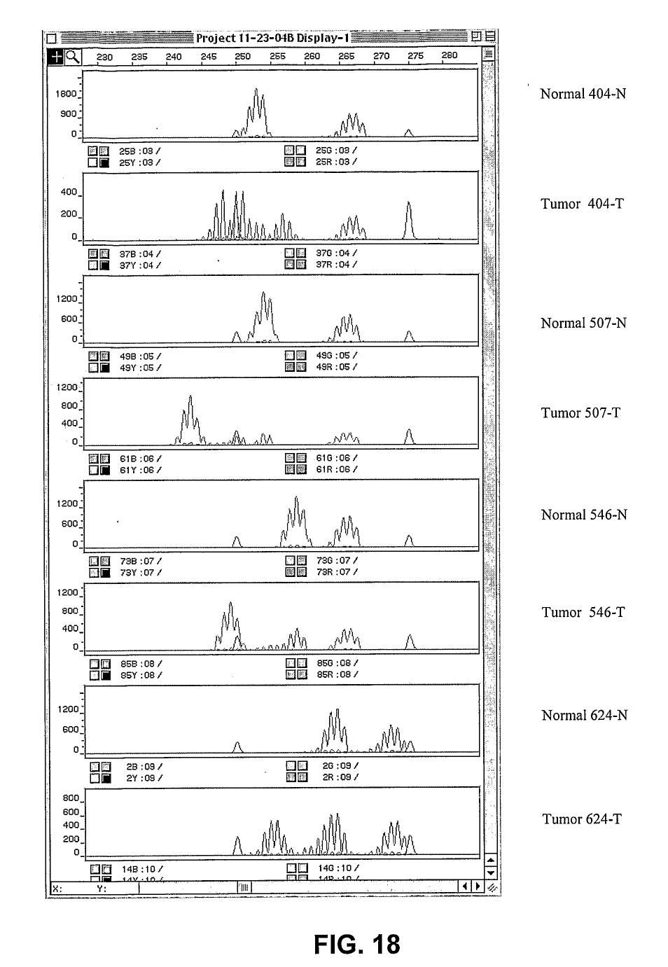

DNA was isolated from numerous colon tumor samples and matching normal tissues using standard methods. The DNA was amplified using primers specific for extended mononucleotide repeat markers (Table 1C) in PCR amplification as described above. The sizes of amplification products for the colon tumor cells were determined and compared with those of the matching normal tissues. New alleles found in tumor samples that were not present in matching normal samples indicted microsatellite instability. The data for two tested extended mononucleotide repeat markers (hBAT-54 and hBAT-60) are presented in FIGS. 17 and 18. These results indicate that the extended mononucleotide repeats are useful in detecting mutations in human tumors.

### G. Method of Distinguishing Mutations from Stutter Artifacts

The ability to distinguish mutations from stutter artifacts is particularly important in genotyping and/or mutational analysis with microsatellite markers (1-6 bp tandem repeats) on single cells or small pools of cells, or their DNA equivalent. The method overcomes a major problem associated with microsatellite analysis with very low amounts of template DNA. During amplification of microsatellite loci, stutter molecules, repeat slippage products formed during PCR, are generated. When formed during the first few PCR cycles, stutter molecules can outnumber the original template molecule(s). The formation of stutter molecules interferes with the ability to distinguish between stutter products and true alleles, thereby confounding interpretation of the data.

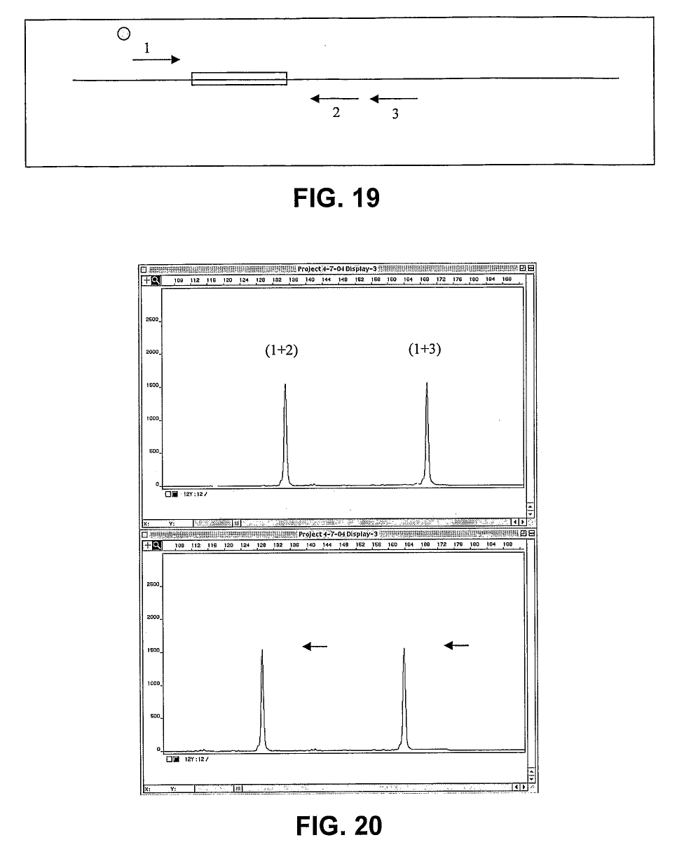

The method relies on coamplification of overlapping amplicons using three primers as illustrated schematically in FIG. 19 and FIG. 20. The method is based on the reduced probability of stutter occurring in exactly the same manner during amplification of two overlapping amplicons. For example, if stutter occurs at a frequency of 0.05, then the chances of stutter occurring in two amplicons is 0.05×0.05, or 2.5 per 1000. This method is thus particularly useful for any type of genotyping or mutational analysis on single or a small number of cells with microsatellite loci in which it is desired to amplify and subsequently identify a few target molecules within a background of non-target molecules. Examples include, but are not limited to, pre-implantation genetic diagnosis (PGD), forensic analysis with very low amounts of DNA, MSI or LOH analysis on single cells or small-pool PCR, and monitoring cell cultures for mutations.

In order to facilitate analysis of amplified target DNA comprising a microsatellite loci from a single cell or using small pool PCR, primers are designed such that the a third primer hybridizes to a region between the members of a primer pair so that two partially overlapping products are formed, each of which contains the repeat locus (FIG. 19). FIG. 20 shows a simulated electropherogram that illustrates the expected results. FIG. 20A shows the sizes of amplification products of a wild type allele. FIG. 20B shows the sizes of amplification products of a mutated allele, which is evidenced by an identical size shift in both amplification products.

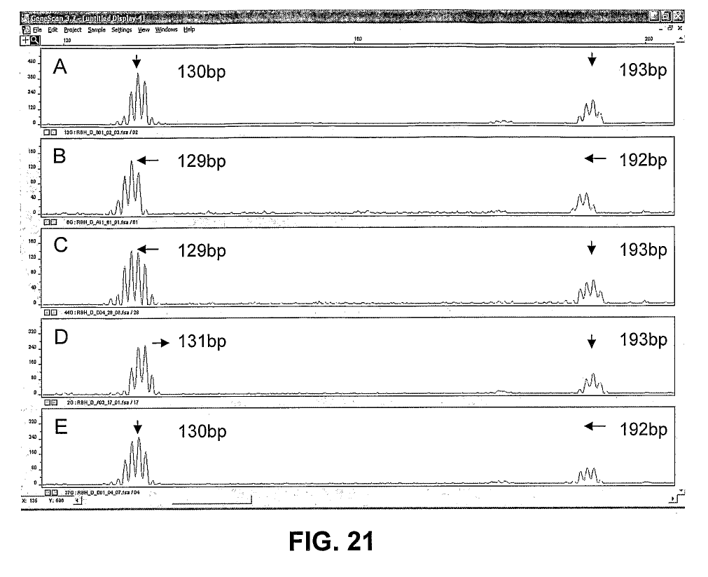

DNA was obtained from mouse embryonic fibroblasts exposed to either 0 Gy or 0.5 Gy iron ions and analyzed for mutations in mBat-26 microsatellite marker using three primers: TCACCATCCATTGCACAGTT (SEQ ID NO:153) labeled with JOE; OH attCTGCGAGAAGGTACTCACCC (SEQ ID NO:167); and OH attACTAGAATCGTACATTGTCCAAAA (SEQ ID NO:168) as shown generally in FIG. 19. When both PCR products were shifted, a sample was determined to have a putative mutation (FIG. 21). Much more commonly, only one PCR product was shifted, which was likely due to stutter occurring in only one of the products during the early rounds of amplification. Thus, this strategy permits mutants to be distinguished from PCR artifacts.

### H. Development of a Reporter System for Evaluating Mutagenicity

In order to provide a reporter system for detecting mutations in response to mutagens, a dual reporter construct will be developed. The construct will include a polynucleotide sequence encoding two different luciferases. Specifically, the construct will contain a first luciferase linked to a second luciferase by an intervening sequence that includes a microsatellite repeat locus having repeats of from 1-6 bases repeated at least 19 times. Preferably, the overall length of the intervening sequence is from about 19 to about 101 bases. The second luciferase will be expressed only if there is a mutation in the intervening sequence that causes the sequence of the second luciferase to be in the proper reading frame. In one embodiment, the construct is represented schematically as follows:

Luciferase (1)-repeat sequence (frame shift)-Luciferase (2)

Luciferase (1) will be constituitively expressed, and Luciferase (2) would be expressed only if a frame shift occurred in the repeat sequence. The ratio of Luciferase (1) to Luciferase (2) expressed would minimize other sources of variation in gene expression and cell viability.

The construct will ideally be designed with a sequence encoding a selectable marker such as an antibiotic resistance marker (e.g., neomycin) fused in-frame to the Luciferase (2). For example, a firefly luciferase (Ffluc) coding sequence can be linked to a sequence encoding Renilla luciferase (Rluc) and a neomycin resistance marker downstream of the Rluc coding sequence, as shown below:

5′-FFluc-repeat sequence (frame shift)-Rluc/neo-3′

To reduce background caused by frame shifts in other regions of the sequence 5′ to repeat sequence, appropriate translation stops will be placed on each side of the repeat sequences as follows:

FFluc-(out-of-frame stop) repeat sequence (in-frame stop)-(frame shift)-Rluc/neo

The construct will be ligated to a suitable vector, preferably an episomal vector having a high copy number. A high copy number vector will enhance the sensitivity of dection by amplifying any mutation that occurs through replication of the episomal vector, thus increasing the rate at which the mutation accumulates. The episomal vector will be capable of replicating in both bacteria (e.g., Eschericia coli) and in mammalian cell lines. Episomal vectors afford simplified clonal purification. Episomal vector systems for mammalian cells have been previous described (Craenenbroeck et al (2000) Eur. J. Biochem. 267:5665-5678; and Conese et al (2004) Gene Therapy 11:1735-1741, each of which is incorporate by reference).

The construct thus produced will be introduced into a cell line or organism will be used as a cellular or in vivo assay for determining the mutagenicity of chemical or biological substances in a manner similar to the Ames test (Ames et al. Science 1972 176:47-49) or Stratagene's Big Blue Mouse (Short et al. Fed. Proc. 1988 8515a; Kohler et al., Proc. Natl. Acad. Sci. USA 1991 88: 7958-7962; and Jakubczak et al., Proc. Natl. Acad. Sci. USA 1996 93: 9073-9078).

Cells containing this reporter vector will be exposed to a mutagen resulting in deletions or insertions in the repeat region and restoration of the reading frame. Subsequent expression of the luciferase coding sequence will increase light signal in a luminescence assay and will be compared to unexposed controls to determine rate of mutation induction.

Each publication or patent application cited herein is incorporated by reference in its entirety.

TABLE 1A

| | | | | |
|---|---|---|---|---|
| Oligo | | | | |
| Synthesis | Marker | | Accession | |
| Number | ID # | Repeat | Number | Primer Sequence |

| | | | | | |
|---|---|---|---|---|---|
| 23098 | mBAT-49 | (A)49 | NT_039456 | GAGTTGGAGGCCAGCTTGGTTTAC | SEQ ID |
| | | | | | NO: 1 |
| 23099 | mBAT-49 | (A)49 | NT_039456 | TGGCTAATCTTCATTGGCTTAACA | SEQ ID |
| | | | | | NO: 2 |
| 23100 | mBAT-50 | (A)50 | NT_039226 | TGTTCTATAAAGCCAATTAACAGA | SEQ ID |
| | | | | | NO: 3 |
| 23101 | mBAT-50 | (A)50 | NT_039226 | CCGAAGTTTTCAATGCCCCATATT | SEQ ID |
| | | | | | NO: 4 |
| 23258 | mBAT-51 | (A)51 | NT_039226 | ACACTGTAGCTGCCTTCCGACACA | SEQ ID |
| | | | | | NO: 5 |
| 23103 | mBAT-51 | (A)51 | NT_039226 | GCAAAGACGGTCCAGCAGTTAAGA | SEQ ID |
| | | | | | NO: 6 |
| 23104 | mBAT- | (A)51 | NT_078407 | CTGCCCAGTGTATGTGACCATCTACTGC | SEQ ID |
| | 51b | | | | NO: 7 |
| 23105 | mBAT- | (A)51 | NT_078407 | GTTGAGGTTAGGTGTAGGCGGCTCTAAT | SEQ ID |
| | 51b | | | | NO: 8 |
| 23106 | mBAT- | (A)51 | NT_078407 | GAAAAGAAGCCATGGGATATAGCC | SEQ ID |
| | 51c | | | | NO: 9 |
| 23107 | mBAT- | (A)51 | NT_078407 | TGCAAGGGTTGAGGTTAGGTGTAG | SEQ ID |
| | 51c | | | | NO: 10 |
| 23108 | mBAT-52 | (A)52 | NT_039413 | TGAATACCCAAAAGCCGCGCTATG | SEQ ID |
| | | | | | NO: 11 |
| 23109 | mBAT-52 | (A)52 | NT_039413 | CGGCCCTCTTCTGGTGTGTCTAAA | SEQ ID |
| | | | | | NO: 12 |
| 23110 | mBAT-53 | (A)53 | NT_039413 | TGATAAACCCTTAGCCAAACTCACTAGA | SEQ ID |
| | | | | | NO: 13 |
| 23111 | mBAT-53 | (A)53 | NT_039413 | CTCTGCACTAAACCCGTTGGTCCT | SEQ ID |
| | | | | | NO: 14 |
| 22155 | mBAT-59 | (A)59 | NT_039624 | GTAATCCCTTTATTCCATTTAGCA | SEQ ID |
| | | | | | NO: 15 |
| 22141 | mBAT-59 | (A)59 | NT_039624 | GGCTCACAACCATCCGTAACAAGA | SEQ ID |
| | | | | | NO: 16 |
| 23259 | mBAT-60 | (A)60 | NT_083168 | GTCAACTTGCCACAAAGTAAAGTC | SEQ ID |
| | | | | | NO: 17 |
| 23113 | mBAT-60 | (A)60 | NT_083168 | CAGAAATCCTACCCATCAATCATT | SEQ ID |
| | | | | | NO: 18 |
| 23260 | mBAT-61 | (A)61 | NT_039226 | CTCCCAAAGTATCCTTCCTAATAG | SEQ ID |
| | | | | | NO: 19 |
| 23115 | mBAT-61 | (A)61 | NT_039226 | TAAGGGCCTTGAATTCCTGATCTT | SEQ ID |
| | | | | | NO: 20 |
| 23116 | mBAT- | (A)61 | NT_078783 | GATGATAGCCTCCAGATACATCCT | SEQ ID |
| | 61c | | | | NO: 21 |
| 23117 | mBAT- | (A)61 | NT_078783 | GCAGACTTTGTGTGGCCCGGTACA | SEQ ID |
| | 61c | | | | NO: 22 |
| 23118 | mBAT-62 | (A)62 | NT_039242 | CCTTTTAGGAACGGTTCGGCCAAT | SEQ ID |
| | | | | | NO: 23 |
| 23119 | mBAT-62 | (A)62 | NT_039242 | AAAGATTATGAAACCAAACTGAGCCTAT | SEQ ID |
| | | | | | NO: 24 |
| 23120 | mBAT- | (A)63 | NT_078817 | CCGACACTGGTTCACCACAACTTA | SEQ ID |
| | 63b | | | | NO: 25 |
| 23121 | mBAT- | (A)63 | NT_078817 | ATCCCCTGGGAAAACCAAATTCAA | SEQ ID |
| | 63b | | | | NO: 26 |
| 22157 | mBAT-64 | (A)64 | NT_039239 | GCCCACACTCCTGAAAACAGTCAT | SEQ ID |
| | | | | | NO: 27 |
| 22141 | mBAT-64 | (A)64 | NT_039239 | CCCTGGTGTGGCAACTTTAAGC | SEQ ID |
| | | | | | NO: 28 |
| 23394 | mBAT-66 | (A)66 | NT_039435 | CACAACCATCCGTAACGAGATCTGACTC | SEQ ID |
| | | | | | NO: 29 |
| 23123 | mBAT-66 | (A)66 | NT_039435 | CCTGAGCCCACTTCATGCGTAACA | SEQ ID |
| | | | | | NO: 30 |
| 22136 | mBAT-67 | (A)67 | AL928868 | CCGACTGCTCTTCCGAAGGTC | SEQ ID |
| | | | | | NO: 31 |
| 22137 | mBAT-67 | (A)67 | AL928868 | TTGCCCATTTATCATCTAGTTCAT | SEQ ID |
| | | | | | NO: 32 |
| 23261 | mBAT-68 | (A)68 | NT_039606 | GAAGGCCCTGCTCTCCTGGTAGAC | SEQ ID |
| | | | | | NO: 33 |
| 23125 | mBAT-68 | (A)68 | NT_039606 | TTTTGTTGGGGCATTGGTTGTTAT | SEQ ID |
| | | | | | NO: 34 |
| 23126 | mBAT-77 | (A)77 | NT_039353 | GCCACCACTGCCCAGCTATGATTG | SEQ ID |
| | | | | | NO: 35 |
| 23131 | mBAT-77 | (A)77 | NT_039353 | CTTGGAAAAGTAAAAGGGGTAAAT | SEQ ID |
| | | | | | NO: 36 |
| 23132 | mBAT-79 | (A)79 | NT_078934 | GTGCAACAAAGACAGGCAATATGT | SEQ ID |
| | | | | | NO: 37 |
| 23133 | mBAT-79 | (A)79 | NT_078934 | GACAGGGGAAAGGGCACACTGACA | SEQ ID |
| | | | | | NO: 38 |
| 23134 | mBAT- | (A)80 | NT_039241 | CTGTACAGCTCATTTGGAGAGTAC | SEQ ID |
| | 80+ | | | | NO: 39 |
| 23135 | mBAT- | (A)80 | NT_039241 | ATTTGTTTGGTATTTCTATTTAGT | SEQ ID |
| | 80+ | | | | NO: 40 |
| 23136 | mBAT-82 | (A)82 | NT_039207 | TCTGATGCCCTCTTCTGGAGTGTC | SEQ ID |
| | | | | | NO: 41 |
| 23137 | mBAT-82 | (A)82 | NT_039207 | CATGGGAGTTAATAGGGTTGTTAG | SEQ ID |
| | | | | | NO: 42 |
| 23138 | mBAT-84 | (A)84 | NT_039180 | ACTTCTGTTTGTCTTTGGGTCAAG | SEQ ID |
| | | | | | NO: 43 |
| 23139 | mBAT-84 | (A)84 | NT_039180 | GCAGACTTTGTGTGCCCCGGTACA | SEQ ID |
| | | | | | NO: 44 |
| 23140 | mBAT-85 | (A)85 | NT_039474 | GCCCCGCCCTGCCCCTCCTAAGTT | SEQ ID |
| | | | | | NO: 45 |
| 23141 | mBAT-85 | (A)85 | NT_039474 | GCTCACAACCATCCGTAACAAGAT | SEQ ID |
| | | | | | NO: 46 |
| 23142 | mBAT- | (A)85 | NT_039609 | ATGACTAGAAGGTGGGAAGATA | SEQ ID |
| | 85b | | | | NO: 47 |
| 23143 | mBAT- | (A)85 | NT_039609 | AAGCAAAGGGGTTCCCGGGAAA | SEQ ID |
| | 85b | | | | NO: 48 |
| 23144 | mBAT- | (A)87 | NT_078297 | GCTTGGGAATGTATGACTTTACCT | SEQ ID |
| | 87+ | | | | NO: 49 |
| 23145 | mBAT- | (A)87 | NT_078297 | CTGACTCATTCGCAAGACGGTCCT | SEQ ID |
| | 87+ | | | | NO: 50 |
| 23146 | mBAT-90 | (A)90 | NT_039305 | TGGAAATGTAAATGGGCTTAATCC | SEQ ID |
| | | | | | NO: 51 |
| 23147 | mBAT-90 | (A)91 | NT_039305 | ATTCTATTCGCTGACTACTTTGTG | SEQ ID |
| | | | | | NO: 52 |
| 23148 | mBAT-97 | (A)97 | NT_078529 | GCCGAATATTTTAATATACATGAT | SEQ ID |
| | | | | | NO: 53 |
| 23149 | mBAT-97 | (A)97 | NT_078529 | GGCCATGACTTTGAGAAGTAAGAG | SEQ ID |
| | | | | | NO: 54 |
| 23150 | mBAT- | (A)209 | NT_078407 | TCTGGCCAGCATTTGCAATCTTTTTGTT | SEQ ID |
| | 209 | | | | NO: 55 |
| 23151 | mBAT- | (A)209 | NT_078407 | CCTCCCCATCTTTATCTAGCAGAGTAAT | SEQ ID |
| | 209 | | | | NO: 56 |

TABLE 1B

| Oligo | | | | | |
|---|---|---|---|---|---|
| Synthesis | Marker | | Accession | | |
| Number | ID# | Repeat | Number | Primer Sequence | |
| 23535 | mBGT-58 | (G)58 | NT_039303 | TGAATTTCTGCCTGCTCAAGTGGATGAT | SEQ ID |
| | | | | | NO: 57 |
| 23536 | mBGT-58 | (G)58 | NT_039303 | GTCGGCGGCGTGGGTGGCGAGCGATTGG | SEQ ID |
| | | | | | NO: 58 |
| 23541 | mBGT-58+ | (G)58 | NT_039472 | TGGGTATCCTAAGTTTCTGGGCTAAGTG | SEQ ID |
| | | | | | NO: 59 |
| 23542 | mBGT-58+ | (G)58 | NT_039472 | GTGGTTGTGGTGGGTCCGCTCTG | SEQ ID |
| | | | | | NO: 60 |
| 23525 | mBGT-66 | (G)66 | NT_039189 | GGCTTATGGATTTATTCTAATGAG | SEQ ID |
| | | | | | NO: 61 |
| 23526 | mBGT-66 | (G)66 | NT_039189 | TGGGCATTCTACAGCTGGTGTCAC | SEQ ID |
| | | | | | NO: 62 |
| 23533 | mBGT-66 | (G)66 | NT_039189 | ACTCGGCTTATGGATTTATTCTAATGAG | SEQ ID |
| | | | | | NO: 63 |
| 23534 | mBGT-66 | (G)66 | NT_039189 | GTAACTTAGTTTCAATGGGCATTCTACA | SEQ ID |
| | | | | | NO: 64 |
| 23547 | mBGT-89+ | (G)89 | NT_039636 | AACAATGGGGAATAGGGCACAGTAAGAC | SEQ ID |
| | | | | | NO: 65 |
| 23548 | mBGT-89+ | (G)89 | NT_039636 | CACCGCCCAACCACCAACACCAC | SEQ ID |
| | | | | | NO: 66 |
| 23539 | mBGT- | (G)116 | NT_039361 | TGTGTGTATGGGTGTATATGAGTATGCG | SEQ ID |
| | 116+ | | | | NO: 67 |
| 23540 | mBGT- | (G)116 | NT_039361 | GTGTAGATGAGGGATGTGGGTATTAGG | SEQ ID |
| | 116+ | | | | NO: 68 |
| 23537 | mBGT- | (G)124 | NT_039359 | CCTTATCTCTTCAGGGGTTCTTAACT | SEQ ID |
| | 124+ | | | | NO: 69 |
| 23538 | mBGT- | (G)124 | NT_039359 | GGGTAGTGTGTGGGTGGTTGGTGTTTGT | SEQ ID |
| | 124+ | | | | NO: 70 |
| 23543 | mBGT-127 | (G)127 | NT_039539 | TATGTACTCCTGATAAGGGAATAGCC | SEQ ID |
| | | | | | NO: 71 |
| 23544 | mBGT-127 | (G)127 | NT_039539 | TGTTAGTATAAAGAGGGGAGTGAATATG | SEQ ID |
| | | | | | NO: 72 |
| 23545 | mBGT- | (G)137 | NT_078778 | CTCTTGCTCCTGCCGCCTCTGCCGATTA | SEQ ID |
| | 137+ | | | | NO: 73 |
| 23546 | mBGT- | (G)137 | NT_078778 | TCCCCTTTTTCTCCCGCGCTCCTGT | SEQ ID |
| | 137+ | | | | NO: 74 |

TABLE 1C

| Oligo | | | | | |
|---|---|---|---|---|---|
| Synthesis | Marker | | Accession | | |
| Number | ID# | Repeat | Number | Primer Sequence | |
| 23158 | hBAT- | (A)48 | AL162713 | TATAATTAGGTCCCAGATCACTTA | SEQ ID |
| | 48 | | | | NO: 75 |
| 23159 | hBAT- | (A)48 | AL162713 | GGCAATGTTTAAAGACATGGATAC | SEQ ID |
| | 48 | | | | NO: 76 |
| 23160 | hBAT- | (A)49 | AC073648 | AAACACAGTGAGACTCCCTATCTA | SEQ ID |
| | 49a | | | | NO: 77 |
| 23161 | hBAT- | (A)49 | AC073648 | ACAGGACAGAGATGGCACGGACAG | SEQ ID |
| | 49a | | | | NO: 78 |
| 23162 | hBAT- | (A)49 | NT_011757 | CTGCTGTTGCATCGCGGCCCAATG | SEQ ID |
| | 49b | | | | NO: 79 |
| 23163 | hBAT- | (A)49 | NT_011757 | AAGAAGCCCCTCTCCTCCGGTCTC | SEQ ID |
| | 49b | | | | NO: 80 |
| 23164 | hBAT- | (A)50 | NT_011669 | AGGCATGGGCAAGGACTTGATGTC | SEQ ID |
| | 50a | | | | NO: 81 |
| 23165 | hBAT- | (A)50 | NT_011669 | CTGGATGTTAGCCGTTTGTCAGAG | SEQ ID |
| | 50a | | | | NO: 82 |
| 23166 | hBAT- | (A)50 | NT_025441 | GGTTTGCTTGAGGCCAGAACTTCA | SEQ ID |
| | 50b | | | | NO: 83 |
| 23167 | hBAT- | (A)50 | NT_025441 | CTCATAGCAGCCTTAAATTACTGA | SEQ ID |
| | 50b | | | | NO: 84 |
| 23168 | hBAT- | (A)51 | BX908732 | AGCCTGGGCGACAGAGCAAGACTC | SEQ ID |
| | 51a | | | | NO: 85 |
| 23169 | hBAT- | (A)51 | BX908732 | CAAGGGCAGCATCATTATGACAAC | SEQ ID |
| | 51a | | | | NO: 86 |
| 23170 | hBAT- | (A)51 | NT_011630 | TGTGTGCAAATTGTGAGGGAGGTAGGTA | SEQ ID |
| | 51b | | | | NO: 87 |
| 23171 | hBAT- | (A)51 | NT_011630 | AGCGGGGTGCGGTGGCTCATATCT | SEQ ID |
| | 51b | | | | NO: 88 |
| 23172 | hBAT- | (A)51 | NT_011786 | GTGAGGCAGGAGAATGGAGAGTAG | SEQ ID |
| | 51c | | | | NO: 89 |
| 23173 | hBAT- | (A)51 | NT_011786 | CTCTGCTACCCGGGTTCAAACAGT | SEQ ID |
| | 51c | | | | NO: 90 |
| 23307 | hBAT- | (A)51 | NT_011903 | GAGGCTGAGGCAGGAGAATGGCGTGAAC | SEQ ID |
| | 51d | | | | NO: 91 |
| 23175 | hBAT- | (A)51 | NT_011903 | CGCTGACGCAGAACCTGAAATTGTGATT | SEQ ID |
| | 51d | | | | NO: 92 |
| 23176 | hBAT- | (A)51 | NT_025965 | AGGTTGCAGTGAGCCAGGATCATA | SEQ ID |
| | 51e | | | | NO: 93 |
| 23289 | hBAT- | (A)51 | NT_025965 | ATCACATCATCTGTCCCACCTAAC | SEQ ID |
| | 51e | | | | NO: 94 |
| 23395 | hBAT- | (A)51 | NT_079573 | TGGGCGACAGAGCGAGACTCCGTC | SEQ ID |
| | 51f | | | | NO: 95 |
| 23179 | hBAT- | (A)51 | NT_079573 | CAGCGGCCCATAAATTCTATGTTA | SEQ ID |
| | 51f | | | | NO: 96 |
| 23181 | hBAT- | (A)52 | NT_011669 | CTAACTTCCCAGCAACTTCCTTTACACT | SEQ ID |
| | 52a | | | | NO: 97 |
| 23182 | hBAT- | (A)52 | NT_011669 | ATTGGGCAGACACTGAACTAGCTT | SEQ ID |
| | 52a | | | | NO: 98 |
| 23183 | hBAT- | (A)52 | NT_025319 | GGGAGAACCTTGCTGTCTTTCAGATAAT | SEQ ID |
| | 52b | | | | NO: 99 |
| 23184 | hBAT- | (A)52 | NT_025319 | AGGGCTCCTGGAATATGGTTGTAC | SEQ ID |
| | 52b | | | | NO: 100 |
| 23298 | hBAT- | (A)53 | AJ549502 | AACCTCCACCTTCCCAGCTCAAGTGACA | SEQ ID |
| | 53a | | | | NO: 101 |
| 23293 | hBAT- | (A)53 | AJ549502 | GGCGACAGCGAGACTCCGTCTCA | SEQ ID |
| | 53a | | | | NO: 102 |
| 23187 | hBAT- | (A)53 | NT_011875 | CTGAGGCAGGAGAATGGCGTGAAC | SEQ ID |
| | 53b | | | | NO: 103 |
| 23188 | hBAT- | (A)53 | NT_011875 | ATGATGCTGGCCTCATAAAAAGAGTTAG | SEQ ID |
| | 53b | | | | NO: 104 |
| 23189 | hBAT- | (A)53 | NT_011896 | TATCCTAGCTTGGCCTGTTTAAGACC | SEQ ID |
| | 53c | | | | NO: 105 |
| 23190 | hBAT- | (A)53 | NT_011896 | TGAGGCAGGAGAATGGCGTGAA | SEQ ID |
| | 53c | | | | NO: 106 |
| 23195 | hBAT- | (A)54 | NT_077819 | TTTAATATACCTGCTGATCAATGATA | SEQ ID |
| | 54 | | | | NO: 107 |
| 23196 | hBAT- | (A)54 | NT_077819 | GACACATGGGATCATAGCAAA | SEQ ID |
| | 54 | | | | NO: 108 |
| 23197 | hBAT- | (A)55 | NT_028405 | TTGGGCGACAGAGCAAGACGACTC | SEQ ID |
| | 55 | | | | NO: 109 |
| 23198 | hBAT- | (A)55 | NT_028405 | ATTTGGTCAGTGGGGGCTCTGTTAAG | SEQ ID |
| | 55 | | | | NO: 110 |
| 23199 | hBAT- | (A)56 | NT_011726 | TCAGCAGCTGAAAGAAATCTGAGTAC | SEQ ID |
| | 56a | | | | NO: 111 |
| 23200 | hBAT- | (A)56 | NT_011726 | GCGATACCCAAAGTCAATAGTC | SEQ ID |
| | 56a | | | | NO: 112 |
| 23201 | hBAT- | (A)56 | NT_011757 | GAAGCTGCAGTAAGCCGAGATTGT | SEQ ID |
| | 56b | | | | NO: 113 |
| 23202 | hBAT- | (A)56 | NT_011757 | GCCCTCTTAACTCCCATGACATTC | SEQ ID |
| | 56b | | | | NO: 114 |
| 23203 | hBAT- | (A)57 | NT_011875 | AGCCTGGGCGACAGAGCGAGTC | SEQ ID |
| | 57 | | | | NO: 115 |
| 23204 | hBAT- | (A)57 | NT_011875 | CTCGGGGCTCGGGAGATGAGTGA | SEQ ID |
| | 57 | | | | NO: 116 |
| 23205 | hBAT- | (A)59 | AC090424 | CAGCCTAGGTAACAGAGCAAGACCTTTG | SEQ ID |
| | 59 | | | | NO: 117 |
| 23206 | hBAT- | (A)59 | AC090424 | GTTTGCGTGATTTGCGTGGACTT | SEQ ID |
| | 59 | | | | NO: 118 |
| 23207 | hBAT- | (A)59 | NT_010783 | CTCCTGCCTCATCCTCCCGAGTA | SEQ ID |
| | 59b | | | | NO: 119 |
| 23208 | hBAT- | (A)59 | NT_010783 | CCGAGATCACGCCACTGCACTCTA | SEQ ID |
| | 59b | | | | NO: 120 |
| 23209 | hBAT- | (A)60 | NT_008183 | TCTCATTTGAGTGGTGGAAGTGACTGGT | SEQ ID |
| | 60a | | | | NO: 121 |
| 23210 | hBAT- | (A)60 | NT_008183 | TATTCTTTCGGGATGTAATCTCT | SEQ ID |
| | 60a | | | | NO: 122 |
| 23211 | hBAT- | (A)60 | NT_022517 | CCCGTCTCTACTAAAAATACTAAAAC | SEQ ID |
| | 60b | | | | NO: 123 |
| 23212 | hBAT- | (A)60 | NT_022517 | AAACCAACAATAAGGCAACCTCTTAGTC | SEQ ID |
| | 60b | | | | NO: 124 |
| 23213 | hBAT- | (A)60 | NT_023089 | TGCCAGAGTAGGGTGGTCCATGGTACTT | SEQ ID |
| | 60c | | | | NO: 125 |
| 23214 | hBAT- | (A)60 | NT_023089 | GCCCAAAATGTGTTTAGTTAGCTTC | SEQ ID |
| | 60c | | | | NO: 126 |
| 23215 | hBAT- | (A)62 | NT_005120 | AGGCTGAAGCAGGAGAATCACTTAAAAC | SEQ ID |
| | 62 | | | | NO: 127 |
| 23216 | hBAT- | (A)62 | NT_005120 | GCCAAGTGTCGCTTGTAATTCTATT | SEQ ID |
| | 62 | | | | NO: 128 |
| 23217 | hBAT- | (A)63 | NT_009775 | GAATCTTGTTTCGGCCTTTGACCTTA | SEQ ID |
| | 63a | | | | NO: 129 |
| 23218 | hBAT- | (A)63 | NT_009775 | CGAGATCACGCCACCGCACTCTAGC | SEQ ID |
| | 63a | | | | NO: 130 |
| 23219 | hBAT- | (A)63 | NT_022184 | AAATCTACCCAGCTCTGTAACGAGAGA | SEQ ID |
| | 63b | | | | NO: 131 |
| 23220 | hBAT- | (A)63 | NT_022184 | AAGCTCTGTTTGGCAAGTGTTAATTGTA | SEQ ID |
| | 63b | | | | NO: 132 |
| 23221 | hBAT- | (A)68 | NT_016354 | TTGGAATGTATTCTCTGGGTTTGGCAGT | SEQ ID |
| | 68a | | | | NO: 133 |
| 23222 | hBAT- | (A)68 | NT_016354 | TTCAGGAGGCTGAGGTGGGAGGATTGT | SEQ ID |
| | 68a | | | | NO: 134 |
| 23223 | hBAT- | (A)68 | NT_079574 | ACCTAGGCAATACCATCTAAGA | SEQ ID |
| | 68b | | | | NO: 135 |
| 23224 | hBAT- | (A)68 | NT_079574 | GTTGCCTGTTCACTCTGATAGTCT | SEQ ID |
| | 68b | | | | NO: 136 |
| 23225 | hBAT- | (A)69 | NT_032977 | AGCCTGGGTGACAGAGCGAGACT | SEQ ID |
| | 69 | | | | NO: 137 |
| 23226 | hBAT- | (A)69 | NT_032977 | TTAGAGTTATTTGTTGGGATGAGAATCT | SEQ ID |
| | 69 | | | | NO: 138 |
| 23227 | hBAT- | (A)72 | NT_037623 | CTGGGCGACAGAGCGAGACTCC | SEQ ID |
| | 72 | | | | NO: 139 |
| 23228 | hBAT- | (A)72 | NT_037623 | TCTCCTGCCTTAGCCTCCCGAGTAGC | SEQ ID |
| | 72 | | | | NO: 140 |
| 23229 | hBAT- | (A)73 | NT_079596 | TCCTCTCCCTAAAAAGCTCCCCCTAAG | SEQ ID |
| | 73 | | | | NO: 141 |
| 23230 | hBAT- | (A)73 | NT_079596 | AGGTCAAGGCTGCGGTAAGCTGTGATCG | SEQ ID |
| | 73 | | | | NO: 142 |
| 23231 | hBAT- | (A)79 | NT_010194 | TCCCCACTTTGTCCTGCACACTCCTACC | SEQ ID |
| | 79 | | | | NO: 143 |
| 23232 | hBAT- | (A)79 | NT_010194 | GGGCGACAGAGCGAGACTCCGTC | SEQ ID |
| | 79 | | | | NO: 144 |
| 23233 | hBAT- | (A)79 | NT_007422 | AAGATTTAATAGACATGCGCAGAACACT | SEQ ID |
| | 83 | | | | NO: 145 |
| 23234 | hBAT- | (A)83 | NT_007422 | CCAGCCTGGGCAAAAGAGCAAGT | SEQ ID |
| | 83 | | | | NO: 146 |
| 23235 | hBAT- | (A)90 | NT_029419 | ACAAACATGAAAAGGCAAATGATAGAAC | SEQ ID |
| | 90 | | | | NO: 147 |
| 23236 | hBAT- | (A)90 | NT_029419 | AGAGGTTGCAGTGAGCCAAGATTGTAG | SEQ ID |
| | 90 | | | | NO: 148 |

TABLE 1D

| Oligo | | | | | |
|---|---|---|---|---|---|
| Synthesis | Marker | | Accession | | |
| Number | ID# | Repeat | Number | Primer Sequence | |
| 23531 | hBGT- | (G)60 | AC002102 | GAGGGATGAAGGGGGACAGATAG | SEQ ID |
| | 60 | | | | NO: 149 |
| 23532 | hBGT- | (G)60 | AC002102 | CATTCTCACTCCACGCCCTCTAT | SEQ ID |
| | 60 | | | | NO: 150 |

TABLE 2

Microsatellite repeat markers for detection of mutations in mice

| | | | | |
|---|---|---|---|---|
| | | GenBank | Chromosomal | |
| Marker | Repeat | Accession | Location | Primers |

| | | | | | |
|---|---|---|---|---|---|
| mBat-24 | (A)24 | U12235 | Chr 7 | CATAGACCCAGTGCTCATCTTCGT | SEQ ID NO: 151 |
| | | | | CATTCGGTGGAAAGCTCTGA | SEQ ID NO: 152 |
| mBat-26 | (A)26 | AF060887 | Chr 11 | TCACCATCCATTGCACAGTT | SEQ ID NO: 153 |
| | | | | CTGCGAGAAGGTACTCACCC | SEQ ID NO: 154 |
| mBat-30 | (A)30 | L24372 | Chr 19 | ATTTGGCTTTCAAGCATCCATA | SEQ ID NO: 155 |
| | | | | GGGAAGACTGCTTAGGGAAGA | SEQ ID NO: 156 |
| mBat-37 | (A)37 | X83972 | Chr 10 | TCTGCCCAAACGTGCTTAAT | SEQ ID NO: 157 |
| | | | | CCTGCCTGGGCTAAAATAGA | SEQ ID NO: 158 |
| mBat-59 | (A)59 | NT_039624 | Chr 16 | GTAATCCCTTTATTCCATTTAGCA | SEQ ID NO: 15 |
| | | | | GGCTCACAACCATCCGTAACAAGA | SEQ ID NO: 16 |
| mBat-64 | (A)64 | NT_039239 | Chr 3 | GCCCACACTCCTGAAAACAGTCAT | SEQ ID NO: 27 |
| | | | | CCCTGGTGTGGCAACTTTAAGC | SEQ ID NO: 28 |
| mBat-67 | (A)67 | AL928868 | Chr 2 | CCGACTGCTCTTCCGAAGGTC | SEQ ID NO: 31 |
| | | | | TTGCCCATTTATCATCTAGTTCAT | SEQ ID NO: 32 |

TABLE 3

Markers for detection of common mitochondrial deletions

| | | | |
|---|---|---|---|
| | | Nucleotide | |
| Marker* | 5′end | position | Primers |

| | | | | |
|---|---|---|---|---|
| mtDNA-1 | F | 12889 bp | TACCATTCCTAACAGGGTTC | SEQ ID NO: 159 |
| | OH | 13341 bp | TTTATGGGTGTAATGCGGTG | SEQ ID NO: 160 |
| mtDNA-2 | F | 8855 bp | AATTCTATTCATCGTCTCGGAAGT | SEQ ID NO: 161 |
| | OH | 13346 bp | TTGAGAGATTTTATGGGTGTAATG | SEQ ID NO: 162 |
| mtDNA-3 | JOE | 89753 bp | TCTCTAGGCCTAGCATATGAAT | SEQ ID NO: 163 |
| | OH | 89873 bp | TTGAAGAAGGTAGATGGCATATTG | SEQ ID NO: 164 |
| mtDNA-4 | F | 16013 bp | CAAAACCCAATCACCTAAGGCTAA | SEQ ID NO: 165 |
| | JOE | 16109 bp | TTTTGGGGTTTGGCATTAAG | SEQ ID NO: 166 |

*Zeng, et al. Journal of Cellular Biochemistry 73: 545-553 (1999)

TABLE 4
Mitochondrial genomic deletions in mice treated with paraquat

| | Young - No Paraquat | | | Old - No Paraquat | | | Old - Paraquat | | |
|---|---|---|---|---|---|---|---|---|---|
| | #1 | #2 | #3 | #4 | #5 | #6 | #7 | #8 | #9 |
| mtDNA-1 | + | + | + | + | + | + | + | + | + |
| (control) | | | | | | | | | |
| mtDNA-2 | − | − | − | − | + | − | + | + | + |
| (deletion) | | | | | | | | | |
| mtDNA-3 | + | + | + | + | + | + | + | + | + |
| (control) | | | | | | | | | |
| mtDNA-4 | + | + | + | + | + | + | + | + | + |
| (control) | | | | | | | | | |

TABLE 5
Mutational analysis of mice treated with paraquat

| | | Young | | | Young w/Paraquat | | | Old | | | Old w/Paraquat | | |
|---|---|---|---|---|---|---|---|---|---|---|---|---|---|
| | Repeat | # | # | Mutation | # | # | Mutation | # | # | Mutation | # | # | Mutation |
| Marker | # | Mutants | Alleles | Freq | Mutants | Alleles | Freq | Mutants | Alleles | Freq | Mutants | Alleles | Freq |
| mBat-24 | | 0 | 182 | 0.000 | 1 | 648 | 0.002 | 0 | 90 | 0.000 | 0 | 318 | 0.000 |
| mBat-26 | | 0 | 222 | 0.000 | 0 | 457 | 0.000 | 0 | 100 | 0.000 | 0 | 340 | 0.000 |
| mBat-30 | | 0 | 230 | 0.000 | 0 | 704 | 0.000 | 0 | 82 | 0.000 | 0 | 344 | 0.000 |
| mBat-37 | | 0 | 244 | 0.000 | 1 | 992 | 0.001 | 0 | 30 | 0.000 | 0 | 306 | 0.000 |
| mBat-49 | | — | — | — | — | — | — | — | — | — | 0 | 194 | 0.000 |
| mBat-51a | | — | — | — | — | — | — | — | — | — | 0 | 240 | 0.000 |
| mBat-51b | | — | — | — | — | — | — | — | — | — | 0 | 186 | 0.000 |
| mBat-52 | | — | — | — | — | — | — | — | — | — | 0 | 44 | 0.000 |
| mBat-61a | | — | — | — | — | — | — | — | — | — | 2 | 428 | 0.005 |
| mBat-59 | | 1 | 364 | 0.003 | 4 | 636 | 0 | 0 | 280 | 0.000 | 6 | 448 | 0.013 |
| mBat-64 | | 0 | 330 | 0.000 | 1 | 443 | 0 | 2 | 362 | 0.006 | 8 | 412 | 0.019 |
| mBat-66 | | 0 | 194 | 0.000 | 3 | 592 | 0 | 1 | 254 | 0.004 | 8 | 448 | 0.018 |
| mBat-67 | | — | — | — | 1 | 334 | 0 | — | — | — | 20 | 394 | 0.051 |

TABLE 6
MSI analysis of Mlh1 and Msh2 deficient intestinal mouse tumors using mononucleotide repeat markers

| | | | Tumor | | | | | | | | |
|---|---|---|---|---|---|---|---|---|---|---|---|
| Sample | Mouse | | Size | Tumor Allele Size Change (bp) | | | | | | | % |
| ID | ID | Genotype | (mm) | mBat-24 | mBat-26 | mBat-30 | mBat-37 | mBat-59 | mBat-64 | mBat-67 | MSI |
| 1N/T | 4934 | Mlh1−/− | 5 | −1 | −2 | 0 | −1 | −6 | −6 | −9 | 86 |
| 2N/T | 4934 | Mlh1−/− | 5 | −1 | 0 | 0 | 0 | nd | nd | nd | 25 |
| 3N/T | 4934 | Mlh1−/− | 5 | −1 | 0 | 0 | −5 | nd | nd | nd | 50 |
| 4N/T | 5461 | Mlh1−/− | 3 | −1 | −1 | −1 | −1 | −1 | −7 | −3 | 100 |
| 5N/T | 5203 | Msh2−/− | 3 | −1 | −1 | −1 | −1 | −2 | −9 | −8 | 100 |
| 6N/T | 5461 | Mlh1−/− | 2 | 0 | −1 | −1 | −1 | −1 | −3 | −1 | 71 |
| 7N/T | 5461 | Mlh1−/− | 2 | 0 | −1 | −2 | −1 | −3 | 0 | −6 | 71 |
| 8N/T | 5734 | Msh2−/− | 2 | −1 | −1 | 0 | −1 | −4 | −3 | −6 | 86 |
| 9N/T | 5734 | Msh2−/− | 2 | −1 | −1 | 0 | −2 | −3 | −5 | −11 | 86 |
| 10N/T | 5734 | Msh2−/− | 2 | −1 | −1 | −3 | −1 | −4 | −7 | −3 | 100 |
| 11N/T | 5734 | Msh2−/− | 1 | 0 | −1 | 0 | 0 | −2 | −2 | −2 | 57 |
| 12N/T | 5278 | Msh2−/− | 0.4 | −1 | −1 | −1 | −1 | 0 | 0 | 0 | 57 |
| 13N/T | 5278 | Msh2−/− | 0.4 | 0 | −1 | −1 | −1 | 0 | 0 | 0 | 42 |

| | | | | | | | |
|---|---|---|---|---|---|---|---|
| Mean Shift (bp)¹ | 1.0 | 1.1 | 1.4 | 1.5 | 3.3 | 5.3 | 5.4 |
| Sensitivity² | 69% | 85% | 53% | 85% | 82% | 72% | 82% |

¹Mean Shift is the average change in size in tumor alleles, excluding zeros.
²Sensitivity is the percent of tumors that displayed instability in a particular marker.

TABLE 7

| | | | | |
|---|---|---|---|---|
| | | | | 5′ |
| Locus | Repeats | Chromosome | Oligonucleotide Sequence | end |

| | | | | | |
|---|---|---|---|---|---|
| DYS393 | (AGAT) | Y | GTG GTC TTC TAC TTG TGT CAA TAC AG | TMR | SEQ ID |
| | | | | | NO: 169 |
| | | | GAA CTC AAG TCC AAA AAA TGA GG | OH | SEQ ID |
| | | | | | NO: 170 |
| DYS390 | (TCTG)/(TCTA) | Y | ATT TAT ATT TTA CAC ATT TTT GGG CC | OH | SEQ ID |
| | | | | | NO: 171 |
| | | | TGA CAG TAA AAT GAA AAC ATT GC | TMR | SEQ ID |
| | | | | | NO: 172 |
| DYS385 | (GAAA) | Y | ATT AGC ATG GGT GAC AGA GCT A | OH | SEQ ID |
| | | | | | NO: 173 |
| | | | CCA ATT ACA TAG TCC TCC TTT C | TMR | SEQ ID |
| | | | | | NO: 174 |
| DYS391 | (TCTA) | Y | TTC AAT CAT ACA CCC ATA TCT GTC | FL | SEQ ID |
| | | | | | NO: 175 |
| | | | ATT ATA GAG GGA TAG GTA GGC AG | OH | SEQ ID |
| | | | | | NO: 176 |
| DYS389I/II | (TCTG)/(TCTA) | Y | CCA ACT CTC ATC TGT ATT ATC TAT G | FL | SEQ ID |
| | | | | | NO: 177 |
| | | | ATT TTA TCC CTG AGT AGC AGA AGA ATG | OH | SEQ ID |
| | | | | | NO: 178 |
| DYS439 | (GATA) | Y | TCG AGT TGT TAT GGT TTT AGG | FL | SEQ ID |
| | | | | | NO: 179 |
| | | | ATT TGG CTT GGA ATT CTT TTA CCC | OH | SEQ ID |
| | | | | | NO: 180 |
| DYS438 | (TTTTC) | Y | TGG GGA ATA GTT GAA CGG TA | JOE | SEQ ID |
| | | | | | NO: 181 |
| | | | ATT GCA ACA AGA GTG AAA CTC CAT T | OH | SEQ ID |
| | | | | | NO: 182 |
| DYS437 | (TCTA)/(TCTG) | Y | ATT GAC TAT GGG CGT GAG TGC AT | OH | SEQ ID |
| | | | | | NO: 183 |
| | | | AGA CCC TGT CAT TCA CAG ATG A | JOE | SEQ ID |
| | | | | | NO: 184 |
| DYS19 | (TAGA) | Y | ACT ACT GAG TTT CTG TTA TAG TGT TTT T | JOE | SEQ ID |
| | | | | | NO: 185 |
| | | | GTC AAT CTC TGC ACC TGG AAA T | OH | SEQ ID |
| | | | | | NO: 186 |
| DYS392 | (TAT) | Y | ATT TAG AGG CAG TCA TCG CAG TG | OH | SEQ ID |
| | | | | | NO: 187 |
| | | | ACC TAC CAA TCC CAT TCC TTA G | JOE | SEQ ID |
| | | | | | NO: 188 |
| NR-21 | (A) | 14 | CGGAGTCGCTGGCACAGTTCTATT | JOE | SEQ ID |
| | | | | | NO: 189 |
| | | | TCGCGTTTACAAACAAGAAAAGTGT | OH | SEQ ID |
| | | | | | NO: 190 |
| BAT-26 | (A) | 2 | TGACTACTTTTGACTTCAGCCAGT | FL | SEQ ID |
| | | | | | NO: 191 |
| | | | AACCATTCAACATTTTTAACCCTT | OH | SEQ ID |
| | | | | | NO: 192 |
| BAT-25 | (A) | 4 | TCGCCTCCAAGAATGTAAGT | JOE | SEQ ID |
| | | | | | NO: 193 |
| | | | ATTTCTGCATTTTAACTATGGCTC | OH | SEQ ID |
| | | | | | NO: 194 |
| NR-24 | (A) | 2 | CCATTGCTGAATTTTACCTC | TMR | SEQ ID |
| | | | | | NO: 195 |
| | | | ATTGTGCCATTGCATTCCAA | OH | SEQ ID |
| | | | | | NO: 196 |
| MONO-27 | (A) | 2 | TGTGAACCACCTATGAATTGCAGA | JOE | SEQ ID |
| | | | | | NO: 197 |
| | | | ATTGCTTGCAGTGAGCAGAGATCGTT | OH | SEQ ID |
| | | | | | NO: 198 |
| Penta C | (AAAAG) | 9 | CATGGCATTGGGGACATGAACACA | TMR | SEQ ID |
| | | | | | NO: 199 |
| | | | CACTGAGCGCTTCTAGGGACTTCT | OH | SEQ ID |
| | | | | | NO: 200 |
| Penta D | (AAAAG) | 21 | CAGCCTAGGTGACAGAGCAAGACA | FL | SEQ ID |
| | | | | | NO: 201 |
| | | | ATTTGCCTAACCTATGGTCATAAC | OH | SEQ ID |
| | | | | | NO: 202 |
| hBAT-51d | (A) | Y | GAGGCTGAGGCAGGAGAATGGCGTGAAC | FL | SEQ ID |
| | | | | | NO: 203 |
| | | | CGCTGACGCAGAACCTGAAATTGTGATT | OH | SEQ ID |
| | | | | | NO: 204 |
| hBAT-53C | (A) | Y | TATCCTAGCTTGGCCTGTTTAAGACC | JOE | SEQ ID |
| | | | | | NO: 205 |
| | | | TGAGGCAGGAGAATGGCGTGAA | OH | SEQ ID |
| | | | | | NO: 206 |
| hBAT-60A | (A) | 8 | TCTCATTTGAGTGGTGGAAGTGACTGGT | JOE | SEQ ID |
| | | | | | NO: 207 |
| | | | TATTCTTTCGGGATGTAATCTCT | OH | SEQ ID |
| | | | | | NO: 208 |
| hBAT-62 | (A) | 2 | AGGCTGAAGCAGGAGAATCACTTAAAAC | JOE | SEQ ID |
| | | | | | NO: 209 |
| | | | GCCAAGTGTCGCTTGTAATTCTATT | OH | SEQ ID |
| | | | | | NO: 210 |
| hBAT-52A | (A) | X | CTAACTTCCCAGCAACTTCCTTTACACT | FL | SEQ ID |
| | | | | | NO: 211 |
| | | | ATTGGGCAGACACTGAACTAGCTT | OH | SEQ ID |
| | | | | | NO: 212 |
| hBAT-59A | (A) | 12 | CAGCCTAGGTAACAGAGCAAGACCTTTG | FL | SEQ ID |
| | | | | | NO: 213 |
| | | | GTTTGCGTGATTTGCGTGGACTT | OH | SEQ ID |
| | | | | | NO: 214 |
| hBAT-56a | (A) | X | TCAGCAGCTGAAAGAAATCTGAGTAC | JOE | SEQ ID |
| | | | | | NO: 215 |
| | | | GCGATACCCAAAGTCAATAGTC | OH | SEQ ID |
| | | | | | NO: 216 |
| hBAT-56b | (A) | X | GAAGCTGCAGTAAGCCGAGATTGT | FL | SEQ ID |
| | | | | | NO: 217 |
| | | | GCCCTCTTAACTCCCATGACATTC | OH | SEQ ID |
| | | | | | NO: 218 |
| D7S3070 | (GATA) | | CATTTCTTCTGCCCCCATGA | | SEQ ID |
| | | | | | NO: 219 |
| | | | attTGACAGCTGAAAAGGTGCAGATG | | SEQ ID |
| | | | | | NO: 220 |
| D7S3046 | (GATA) | | GAGGAGACAGCCAGGGATATA | | SEQ ID |
| | | | | | NO: 221 |
| | | | attTCTCTATAACCTCTCTCCCTATCT | | SEQ ID |
| | | | | | NO: 222 |
| D7S1808 | (GGAA) | | GGAGGAAAAGTCTTAAACGTGAAT | | SEQ ID |
| | | | | | NO: 223 |
| | | | attGGCCTTGATGTGTTTGTTACT | | SEQ ID |
| | | | | | NO: 224 |
| D10S1426 | (GATA) | | GCCGATCCTGAAGCAATAGC | | SEQ ID |
| | | | | | NO: 225 |
| | | | attCCCCTTGGTGGTGTCATCCT | | SEQ ID |
| | | | | | NO: 226 |
| D3S2432 | (GATA) | | GTTTGCATGTGAACAGGTCA | | SEQ ID |
| | | | | | NO: 227 |
| | | | attGGCAGGCAGGTAGATAGACA | | SEQ ID |
| | | | | | NO: 228 |
| FGA | (TTTC) | 4 | GGCTGCAGGGCATAACATTA | TMR | SEQ ID |
| | | | | | NO: 229 |
| | | | ATTCTATGACTTTGCGCTTCAGGA | OH | SEQ ID |
| | | | | | NO: 230 |
| TPOX | (AATG) | 2 | GCACAGAACAGGCACTTAGG | OH | SEQ ID |
| | | | | | NO: 231 |
| | | | CGCTCAAACGTGAGGTTG | TMR | SEQ ID |
| | | | | | NO: 232 |
| D8S1179 | (TCTA) | 8 | ATTGCAACTTATATGTATTTTTGTATTTCATG | OH | SEQ ID |
| | | | | | NO: 233 |
| | | | ACCAAATTGTGTTCATGAGTATAGTTTC | TMR | SEQ ID |
| | | | | | NO: 234 |
| vWA | (TCTA) | 12 | GCCCTAGTGGATGATAAGAATAATCAGTATGTG | OH | SEQ ID |
| | | | | | NO: 235 |
| | | | GGACAGATGATAAATACATAGGATGGATGG | TMR | SEQ ID |
| | | | | | NO: 236 |
| Amelogenin | | X | CCCTGGGCTCTGTAAAGAA | TMR | SEQ ID |
| | | | | | NO: 237 |
| | | | ATCAGAGCTTAAACTGGGAAGCTG | OH | SEQ ID |
| | | | | | NO: 238 |
| Penta E | (AAAGA) | 15 | ATTACCAACATGAAAGGGTACCAATA | OH | SEQ ID |
| | | | | | NO: 239 |
| | | | TGGGTTATTAATTGAGAAAACTCCTTACAATTT | FL | SEQ ID |
| | | | | | NO: 240 |
| D18S51 | (AGAA) | 18 | TTCTTGAGCCCAGAAGGTTA | FL | SEQ ID |
| | | | | | NO: 241 |
| | | | ATTCTACCAGCAACAACACAAATAAAC | OH | SEQ ID |
| | | | | | NO: 242 |
| D21S11 | (TCTA) | 21 | ATATGTGAGTCAATTCCCCAAG | OH | SEQ ID |
| | | | | | NO: 243 |
| | | | TGTATTAGTCAATGTTCTCCAGAGAC | FL | SEQ ID |
| | | | | | NO: 244 |
| TH01 | (AATG) | 11 | GTGATTCCCATTGGCCTGTTC | FL | SEQ ID |
| | | | | | NO: 245 |
| | | | ATTCCTGTGGGCTGAAAAGCTC | OH | SEQ ID |
| | | | | | NO: 246 |
| D3S1358 | (TCTA) | 3 | ACTGCAGTCCAATCTGGGT | OH | SEQ ID |
| | | | | | NO: 247 |
| | | | ATGAAATCAACAGAGGCTTGC | FL | SEQ ID |
| | | | | | NO: 248 |
| Penta D | (AAAGA) | 21 | GAAGGTCGAAGCTGAAGTG | JOE | SEQ ID |
| | | | | | NO: 249 |
| | | | ATTAGAATTCTTTAATCTGGACACAAG | OH | SEQ ID |
| | | | | | NO: 250 |
| CSF1PO | (AGAT) | 5 | CCGGAGGTAAAGGTGTCTTAAAGT | JOE | SEQ ID |
| | | | | | NO: 251 |
| | | | ATTTCCTGTGTCAGACCCTGTT | OH | SEQ ID |
| | | | | | NO: 252 |
| D16S539 | (GATA) | 16 | GGGGGTCTAAGAGCTTGTAAAAAG | OH | SEQ ID |
| | | | | | NO: 253 |
| | | | GTTTGTGTGTGCATCTGTAAGCATGTATC | JOE | SEQ ID |
| | | | | | NO: 254 |
| D7S820 | (GATA) | 7 | ATGTTGGTCAGGCTGACTATG | JOE | SEQ ID |
| | | | | | NO: 255 |
| | | | GATTCCACATTTATCCTCATTGAC | OH | SEQ ID |
| | | | | | NO: 256 |
| D13S317 | (TATC) | 13 | ATTACAGAAGTCTGGGATGTGGAGGA | OH | SEQ ID |
| | | | | | NO: 257 |
| | | | GGCAGCCCAAAAAGACAGA | JOE | SEQ ID |
| | | | | | NO: 258 |
| D5S818 | (AGAT) | 5 | GGTGATTTTCCTCTTTGGTATCC | OH | SEQ ID |
| | | | | | NO: 259 |
| | | | AGCCAGAGTTTACAACATTTGTATCT | JOE | SEQ ID |
| | | | | | NO: 260 |

1. A method for monitoring an organism or cell population for exposure to a mutagen comprising:
(a) obtaining a first DNA sample from the organism or cell population, the first DNA sample comprising a set of at least one microsatellite locus selected from the group consisting of mononucleotide repeat loci having at least 38 repeats; Y chromosome short tandem repeats of 1-6 bp; and A-rich short tandem repeats having repeating units selected from the group consisting of AAAAG, AAAAC, and AAAAT;
(b) contacting the first DNA sample with a first primer and a second primer that hybridize to a first DNA sequence and a second DNA sequence, respectively, the first and second DNA sequences flanking or partially overlapping the microsatellite locus, under conditions that allow amplification of the microsatellite locus to form a first amplification product;
(c) determining the size of the first amplification product; and
(d) comparing the size of the first amplification product to the expected size of the amplification product, a difference between the size of first amplification product and the expected size of the amplification product being indicative of exposure to a mutagen.

2. The method of claim 1, wherein the expected size of the amplification product of step (d) is determined by:
(e) obtaining a control DNA sample from the organism or cell population prior to obtaining the first DNA sample of step (a), the control DNA sample comprising the set of at least one microsatellite locus of step (a);
(f) contacting the control DNA sample with the first primer and second primer of step (b) under conditions that allow amplification of the microsatellite locus of step (e) to form a second amplification product;
(g) determining the size of the second amplification product, wherein the size of the second amplification product is the expected size of the amplification product of step (d).

3. The method of claim 1, wherein the set of at least one microsatellite locus comprises at least one locus selected from the group of loci described in Table 1A-D.

4. The method of claim 1, wherein the set of at least one microsatellite locus comprises at least one of Y chromosome short tandem repeats of 1-6 bp described in Table 7.

5. The method of claim 1, wherein the set of at least one microsatellite locus comprises at least one locus selected from the group of loci consisting of DYS391, DYS389 I, DYS389 II, DYS438, DYS437, DYS19, DYS392, DYS393, DYS390, and DYS385.

6. The method of claim 1, wherein the set of at least one microsatellite locus comprises at least one locus selected from the group consisting of mbat-59, mbat-61a, mbat-64, mbat-66, mbat-67, hBAT-60a, hBAT-51d, hBAT-53c, hBAT59a, and hBAT-62.

7. The method of claim 1, wherein the set of at least one microsatellite locus comprises at least one of Penta C and Penta D.

8. The method of claim 1, wherein the mutagen is radiation, a free radical or reactive oxygen species, a substance that causes a free radical or reactive oxygen species to form, or an environmental condition that causes a free radical or reactive oxygen species to form.

9. A method for evaluating the mutagenicity of an agent comprising:
(a) exposing an organism or cell culture to the agent;
(b) obtaining a first DNA sample from the organism or cell culture of step (a), the first DNA sample comprising a set of at least one microsatellite locus selected from the group of loci consisting of: mononucleotide repeat loci having at least 38 repeats; Y chromosome short tandem repeat of 1-6 bp; and A-rich short tandem repeats having repeating units selected from the group consisting of AAAAG, AAAAG, and AAAAT;
(c) contacting the first DNA sample with a first primer and a second primer that hybridize to a first DNA sequence and second DNA sequence, respectively, the first and second DNA sequences flanking or partially overlapping the microsatellite locus, under conditions that allow amplification of the locus to form a first amplification product;
(d) determining the size of the first amplification product; and
(e) comparing the size of the first amplification product to the expected size of the amplification product, wherein a difference between the size of the first amplification product and expected size of the amplification product is indicative of mutagenicity.

10. The method of claim 9, wherein the expected size of the amplification product of step (e) is determined by:
(f) obtaining a control DNA sample selected from the group consisting of: a DNA sample obtained from the organism or cell culture prior to obtaining the first DNA sample of step (b); and a DNA sample obtained from a second organism or cell culture not exposed to the agent, the control DNA sample comprising set of at least one microsatellite locus of step (b);
(g) contacting the control DNA sample with the first and second primers of step (b) under conditions that allow amplification of the set of at least one microsatellite locus to form a second amplification product;
(h) determining the size of the second amplification product, wherein the size of the second amplification product is the expected size of the amplification product of step (e).

11. The method of claim 10, wherein the control DNA sample is obtained from the organism or cell culture prior to exposure to the agent.

12. The method of claim 9, wherein the set of at least one microsatellite locus comprises at least one locus selected from the group of loci described in Table 1A-D.

13. The method of claim 9, wherein the set of at least one microsatellite locus comprises at least one locus selected from the group of Y chromosome short tandem repeats of 1-6 bp described in Table 7.

14. The method of claim 9, wherein the set of at least one microsatellite locus comprises at least one locus selected from the group of loci consisting of DYS391, DYS389 I, DYS389 II, DYS438, DYS437, DYS19, DYS392, DYS393, DYS390, and DYS385.

15. The method of claim 9, wherein the set of at least one microsatellite locus comprises at least one locus selected from the group consisting of mbat-59, mbat-61a, mbat-64, mbat-66, mbat-67, hBAT-60a, hBAT-51d, hBAT-53c, hBAT59a, and hBAT-62.

16. The method of claim 9, wherein the set of at least one microsatellite locus comprises at least one of Penta C and Penta D.

17. A method of detecting microsatellite instability in a human putative cancerous or precancerous cell or tumor comprising:
(a) obtaining a first DNA sample from the putative cancerous or precancerous cell or tumor, the first DNA sample comprising a set of at least one microsatellite locus selected from the group consisting of mononucleotide repeat loci having at least 41 repeats and Y chromosome short tandem repeats of 1-6 bp;
(b) contacting the first DNA sample with a first primer and a second primer that hybridize to a first DNA sequence and a second DNA sequence, respectively, the first and second DNA sequences flanking or partially overlapping the microsatellite locus, under conditions that allow amplification of the microsatellite locus to form a first amplification product;
(c) determining the size of the first amplification product; and
(d) comparing the size of the first amplification product to the expected size of the amplification product, a difference between the size of first amplification product and the expected size of the amplification product being indicative of microsatellite instability.

18. The method of claim 17, wherein the expected size of the amplification product of step (d) is determined by:
(e) obtaining a control DNA sample from a normal cell comprising a set of at least one microsatellite locus of step (a);
(f) contacting the control DNA sample with the first and second primers of step (g) under conditions that allow amplification of the set of at least one microsatellite locus to form a second amplification product;
(h) determining the size of the second amplification product, wherein the size of the second amplification product is the expected size of the amplification product of step (d).

19. The method of claim 17, wherein the set of at least one microsatellite locus comprises at least one locus selected from the group of loci described in Table 1C-D.

20. The method of claim 17, wherein the set of at least one microsatellite locus comprises at least one locus selected from the group of Y chromosome short tandem repeats of 1-6 bp described in Table 7.

21. The method of claim 17, wherein the set of at least one microsatellite locus comprises at least one locus selected from the group of loci consisting of DYS391, DYS389 I, DYS389 II, DYS438, DYS437, DYS19, DYS392, DYS393, DYS390, and DYS385.

22. The method of claim 17, wherein the set of at least one microsatellite locus comprises at least one locus selected from the group consisting of hBAT-60a, hBAT-51d, hBAT-53c, hBAT59a, and hBAT-62.

23. A method of detecting microsatellite instability in a mouse putative cancerous or precancerous cell or tumor comprising:
(a) obtaining a first DNA sample from the putative cancerous or precancerous cell or tumor, the first DNA sample comprising a set of at least one mononucleotide repeat locus having at least 48 repeats;
(b) contacting the first DNA sample with a first primer and a second primer that hybridize to a first DNA sequence and a second DNA sequence, respectively, the first and second DNA sequences flanking or partially overlapping the mononucleotide repeat, under conditions that allow amplification of the mononucleotide repeat to form a first amplification product;
(c) determining the size of the first amplification product; and
(d) comparing the size of the first amplification product to the expected size of the amplification product, a difference between the size of first amplification product and the expected size of the amplification product being indicative of microsatellite instability.

24. The method of claim 23, wherein the expected size of the amplification product of step (d) is determined by:
(e) obtaining a control DNA sample from a normal cell comprising set of at least one mononucleotide repeat locus of step (a);
(f) contacting control DNA sample with the first and second primers of step (b) under conditions that allow amplification of the set of at least one mononucleotide repeat locus to form a second amplification product;
(h) determining the size of the second amplification product, wherein the size of the second amplification product is the expected size of the amplification of step (d).

25. The method of claim 23, wherein the set of at least one microsatellite locus comprises at least one locus selected from the group of loci described in Table 1A-B.

26. The method of claim 23 wherein the set of at least one microsatellite locus comprises at least one locus selected from the group consisting of mbat-59, mbat-61a, mbat-64, mbat-66, and mbat-67.

27. The method of claim 26, wherein the set at least one microsatellite locus comprises at mbat-59, mbat-64, and mbat-67, and further comprises mBat-26 and mBat-37.

28. A method for detecting a mutation in a microsatellite locus comprising:
(a) obtaining a first DNA sample from a human cell line or individual, the DNA sample comprising a set of at least one microsatellite locus selected from mononucleotide repeat loci having at least 41 repeats;
(b) contacting the sample with a first primer and a second primer that hybridize to a first DNA sequence and second DNA sequence, respectively, the first and second DNA sequences flanking or partially overlapping the microsatellite locus, under conditions that allow amplification of the at least one microsatellite locus to form a first amplification product;
(c) determining the size of the first amplification product; and
(d) comparing the size of the first amplification product to the expected size of the amplification product, a difference between the size of the first amplification product and the expected size of the amplification product being indicative of a mutation in the microsatellite repeat locus.

29. The method of claim 28, wherein the set of at least one microsatellite locus comprises at least one locus selected from the group of loci described in Table 1C-D.

30. The method of claim 28, wherein the set of at least one microsatellite locus comprises at least one locus selected from the group consisting of hBAT-60a, hBAT-51d, hBAT-53c, hBAT59a, and hBAT-62.

31. A method for detecting a mutation in a microsatellite locus comprising:
(a) obtaining a first DNA sample from a mouse or mouse cell line, the DNA sample comprising a set of at least one mononucleotide repeat locus having at least 48 repeats;
(b) contacting the sample with a first primer and a second primer that hybridize to a first DNA sequence and second DNA sequence, respectively, the first and second DNA sequences flanking or partially overlapping the microsatellite locus, under conditions that allow amplification of the at least one microsatellite locus to form a first amplification product;
(c) determining the size of the first amplification product; and
(d) comparing the size of the first amplification product to the expected size of the amplification product, a difference between the size of the first amplification product and the expected size of the amplification product being indicative of a mutation in the microsatellite repeat locus.

32. The method of claim 31, wherein the set of at least one microsatellite locus comprises at least one locus selected from the group of loci described in Table 1A-B.

33. The method of claim 31, wherein the set of at least one microsatellite locus comprises at least one locus selected from the group consisting of mbat-59, mbat-61a, mbat-64, mbat-66, and mbat-67.

34. A method for distinguishing between a mutation and an artifact comprising:
(a) contacting a sample comprising a target DNA sequence comprising a mono-, di- tri-, tetra-, penta-, or hexanucleotide repeat locus with a first primer, a second primer, and a third primer, the first primer hybridizing to a first sequence and the second primer hybridizing to a second sequence, the first and second sequences flanking or partially overlapping the target DNA sequence, and the third primer hybridizing to a third sequence between the first and second sequences, under conditions that allow amplification of the target DNA between the first and second primers to form a first amplification product and amplification of the target DNA between the first and third primers to form a second amplification product;
(b) determining the sizes of the first and second amplification products; and
(c) comparing the size difference between the measured and expected size of the first amplification product with the size difference between the measured and expected size of the second amplification product, an equivalent size difference in the first and second amplification products relative to their respective expected sizes indicating a mutation.

35-49. (canceled)

50. A method for evaluating mutagenicity of an agent comprising:
(a) exposing a cell or organism comprising the construct of claim 36 to an agent; and
(b) detecting a change in expression of the reporter marker, wherein the change in expression of the reporter marker is indicative of the mutagenicity of the agent.

51. (canceled)


SEQUENCE LISTING (260 sequences)

SEQ ID NO: 1 - DNA, 24 bp, Mus sp.
gagttggaggccagcttggtttac

SEQ ID NO: 2 - DNA, 24 bp, Mus sp.
tggctaatcttcattggcttaaca

SEQ ID NO: 3 - DNA, 24 bp, Mus sp.
tgttctataaagccaattaacaga

SEQ ID NO: 4 - DNA, 24 bp, Mus sp.
ccgaagttttcaatgccccatatt

SEQ ID NO: 5 - DNA, 24 bp, Mus sp.
acactgtagctgccttccgacaca

SEQ ID NO: 6 - DNA, 24 bp, Mus sp.
gcaaagacggtccagcagttaaga

SEQ ID NO: 7 - DNA, 28 bp, Mus sp.
ctgcccagtgtatgtgaccatctactgc

SEQ ID NO: 8 - DNA, 28 bp, Mus sp.
gttgaggttaggtgtaggcggctctaat

SEQ ID NO: 9 - DNA, 24 bp, Mus sp.
gaaaagaagccatgggatatagcc

SEQ ID NO: 10 - DNA, 24 bp, Mus sp.
tgcaagggttgaggttaggtgtag

SEQ ID NO: 11 - DNA, 24 bp, Mus sp.
tgaatacccaaaagccgcgctatg

SEQ ID NO: 12 - DNA, 24 bp, Mus sp.
cggccctcttctggtgtgtctaaa

SEQ ID NO: 13 - DNA, 28 bp, Mus sp.
tgataaacccttagccaaactcactaga

SEQ ID NO: 14 - DNA, 24 bp, Mus sp.
ctctgcactaaacccgttggtcct

SEQ ID NO: 15 - DNA, 24 bp, Mus sp.
gtaatccctttattccatttagca

SEQ ID NO: 16 - DNA, 24 bp, Mus sp.
ggctcacaaccatccgtaacaaga

SEQ ID NO: 17 - DNA, 24 bp, Mus sp.
gtcaacttgccacaaagtaaagtc

SEQ ID NO: 18 - DNA, 24 bp, Mus sp.
cagaaatcctacccatcaatcatt

SEQ ID NO: 19 - DNA, 24 bp, Mus sp.
ctcccaaagtatccttcctaatag

SEQ ID NO: 20 - DNA, 24 bp, Mus sp.
taagggccttgaattcctgatctt

SEQ ID NO: 21 - DNA, 24 bp, Mus sp.
gatgatagcctccagatacatcct

SEQ ID NO: 22 - DNA, 24 bp, Mus sp.
gcagactttgtgtggcccggtaca

SEQ ID NO: 23 - DNA, 24 bp, Mus sp.
ccttttaggaacggttcggccaat

SEQ ID NO: 24 - DNA, 28 bp, Mus sp.
aaagattatgaaaccaaactgagcctat

SEQ ID NO: 25 - DNA, 24 bp, Mus sp.
ccgacactggttcaccacaactta

SEQ ID NO: 26 - DNA, 24 bp, Mus sp.
atcccctgggaaaaccaaattcaa

SEQ ID NO: 27 - DNA, 24 bp, Mus sp.
gcccacactcctgaaaacagtcat

SEQ ID NO: 28 - DNA, 22 bp, Mus sp.
ccctggtgtggcaactttaagc

SEQ ID NO: 29 - DNA, 28 bp, Mus sp.
cacaaccatccgtaacgagatctgactc

SEQ ID NO: 30 - DNA, 24 bp, Mus sp.
cctgagcccacttcatgcgtaaca

SEQ ID NO: 31 - DNA, 21 bp, Mus sp.
ccgactgctcttccgaaggtc

SEQ ID NO: 32 - DNA, 24 bp, Mus sp.
ttgcccatttatcatctagttcat

SEQ ID NO: 33 - DNA, 24 bp, Mus sp.
gaaggccctgctctcctggtagac

SEQ ID NO: 34 - DNA, 24 bp, Mus sp.
ttttgttggggcattggttgttat

SEQ ID NO: 35 - DNA, 24 bp, Mus sp.
gccaccactgcccagctatgattg

SEQ ID NO: 36 - DNA, 24 bp, Mus sp.
cttggaaaagtaaaaggggtaaat

SEQ ID NO: 37 - DNA, 24 bp, Mus sp.
gtgcaacaaagacaggcaatatgt

SEQ ID NO: 38 - DNA, 24 bp, Mus sp.
gacaggggaaagggcacactgaca

SEQ ID NO: 39 - DNA, 24 bp, Mus sp.
ctgtacagctcatttggagagtac

SEQ ID NO: 40 - DNA, 24 bp, Mus sp.
atttgtttggtatttctatttagt

SEQ ID NO: 41 - DNA, 24 bp, Mus sp.
tctgatgccctcttctggagtgtc

SEQ ID NO: 42 - DNA, 24 bp, Mus sp.
catgggagttaatagggttgttag

SEQ ID NO: 43 - DNA, 24 bp, Mus sp.
acttctgtttgtctttgggtcaag

SEQ ID NO: 44 - DNA, 24 bp, Mus sp.
gcagactttgtgtgccccggtaca

SEQ ID NO: 45 - DNA, 24 bp, Mus sp.
gccccgccctgcccctcctaagtt

SEQ ID NO: 46 - DNA, 24 bp, Mus sp.
gctcacaaccatccgtaacaagat

SEQ ID NO: 47 - DNA, 22 bp, Mus sp.
atgactagaaggtgggaagata

SEQ ID NO: 48 - DNA, 22 bp, Mus sp.
aagcaaaggggttcccgggaaa

SEQ ID NO: 49 - DNA, 24 bp, Mus sp.
gcttgggaatgtatgactttacct

SEQ ID NO: 50 - DNA, 24 bp, Mus sp.
ctgactcattcgcaagacggtcct

SEQ ID NO: 51 - DNA, 24 bp, Mus sp.
tggaaatgtaaatgggcttaatcc

SEQ ID NO: 52 - DNA, 24 bp, Mus sp.
attctattcgctgactactttgtg

SEQ ID NO: 53 - DNA, 24 bp, Mus sp.
gccgaatattttaatatacatgat

SEQ ID NO: 54 - DNA, 24 bp, Mus sp.
ggccatgactttgagaagtaagag

SEQ ID NO: 55 - DNA, 28 bp, Mus sp.
tctggccagcatttgcaatctttttctt

SEQ ID NO: 56 - DNA, 28 bp, Mus sp.
cctccccatctttatctagcagagtaat

SEQ ID NO: 57 - DNA, 28 bp, Mus sp.
tgaatttctgcctgctcaagtggatgat

SEQ ID NO: 58 - DNA, 28 bp, Mus sp.
gtcggcggcgtgggtggcgagcgattgg

SEQ ID NO: 59 - DNA, 28 bp, Mus sp.
tgggtatcctaagtttctgggctaagtg

SEQ ID NO: 60 - DNA, 23 bp, Mus sp.
gtggttgtggtgggtccgctctg

SEQ ID NO: 61 - DNA, 24 bp, Mus sp.
ggcttatggatttattctaatgag

SEQ ID NO: 62 - DNA, 24 bp, Mus sp.
tgggcattctacagctggtgtcac

SEQ ID NO: 63 - DNA, 28 bp, Mus sp.
actcggcttatggatttattctaatgag

SEQ ID NO: 64 - DNA, 28 bp, Mus sp.
gtaacttagtttcaatgggcattctaca

SEQ ID NO: 65 - DNA, 28 bp, Mus sp.
aacaatggggaatagggcacagtaagac

SEQ ID NO: 66 - DNA, 23 bp, Mus sp.
caccgcccaaccaccaacaccac

SEQ ID NO: 67 - DNA, 28 bp, Mus sp.
tgtgtgtatgggtgtatatgagtatgcg

SEQ ID NO: 68 - DNA, 27 bp, Mus sp.
gtgtagatgagggatgtgggtattagg

SEQ ID NO: 69 - DNA, 26 bp, Mus sp.
ccttatctcttcaggggttcttaact

SEQ ID NO: 70 - DNA, 28 bp, Mus sp.
gggtagtgtgtgggtggttggtgtttgt

SEQ ID NO: 71 - DNA, 26 bp, Mus sp.
tatgtactcctgataagggaatagcc

SEQ ID NO: 72 - DNA, 28 bp, Mus sp.
tgttagtataaagaggggagtgaatatg

SEQ ID NO: 73 - DNA, 28 bp, Mus sp.
ctcttgctcctgccgcctctgccgatta

SEQ ID NO: 74 - DNA, 25 bp, Mus sp.
tcccctttttctcccgcgctcctgt

SEQ ID NO: 75 - DNA, 24 bp, Homo sapiens
tataattaggtcccagatcactta

SEQ ID NO: 76 - DNA, 24 bp, Homo sapiens
ggcaatgtttaaagacatggatac

SEQ ID NO: 77 - DNA, 24 bp, Homo sapiens
aaacacagtgagactccctatcta

SEQ ID NO: 78 - DNA, 24 bp, Homo sapiens
acaggacagagatggcacggacag

SEQ ID NO: 79 - DNA, 24 bp, Homo sapiens
ctgctgttgcatcgcggcccaatg

SEQ ID NO: 80 - DNA, 24 bp, Homo sapiens
aagaagcccctctcctccggtctc

SEQ ID NO: 81 - DNA, 24 bp, Homo sapiens
aggcatgggcaaggacttgatgtc

SEQ ID NO: 82 - DNA, 24 bp, Homo sapiens
ctggatgttagccgtttgtcagag

SEQ ID NO: 83 - DNA, 24 bp, Homo sapiens
ggtttgcttgaggccagaacttca

SEQ ID NO: 84 - DNA, 24 bp, Homo sapiens
ctcatagcagccttaaattactga

SEQ ID NO: 85 - DNA, 24 bp, Homo sapiens
agcctgggcgacagagcaagactc

SEQ ID NO: 86 - DNA, 24 bp, Homo sapiens
caagggcagcatcattatgacaac

SEQ ID NO: 87 - DNA, 28 bp, Homo sapiens
tgtgtgcaaattgtgagggaggtaggta

SEQ ID NO: 88 - DNA, 24 bp, Homo sapiens
agcggggtgcggtggctcatatct

SEQ ID NO: 89 - DNA, 24 bp, Homo sapiens
ctgaggcaggagaatggagagtag

SEQ ID NO: 90 - DNA, 24 bp, Homo sapiens
ctctgctacccgggttcaaacagt

SEQ ID NO: 91 - DNA, 28 bp, Homo sapiens
gaggctgaggcaggagaatggcgtgaac

SEQ ID NO: 92 - DNA, 28 bp, Homo sapiens
cgctgacgcagaacctgaaattgtgatt

SEQ ID NO: 93 - DNA, 24 bp, Homo sapiens
aggttgcagtgagccaggatcata

SEQ ID NO: 94 - DNA, 24 bp, Homo sapiens
atcacatcatctgtcccacctaac

SEQ ID NO: 95 - DNA, 24 bp, Homo sapiens
tgggcgacagagcgagactccgtc

SEQ ID NO: 96 - DNA, 24 bp, Homo sapiens
cagcggcccataaattctatgtta

SEQ ID NO: 97 - DNA, 28 bp, Homo sapiens
ctaacttcccagcaacttcctttacact

SEQ ID NO: 98 - DNA, 24 bp, Homo sapiens
attgggcagacactgaactagctt

SEQ ID NO: 99 - DNA, 28 bp, Homo sapiens
gggagaaccttgctgtctttcagataat

SEQ ID NO: 100 - DNA, 24 bp, Homo sapiens
agggctcctggaatatggttgtac

SEQ ID NO: 101 - DNA, 28 bp, Homo sapiens
aacctccaccttcccagctcaagtgaca

SEQ ID NO: 102 - DNA, 23 bp, Homo sapiens
ggcgacagcgagactccgtctca

SEQ ID NO: 103 - DNA, 24 bp, Homo sapiens
ctgaggcaggagaatggcgtgaac

SEQ ID NO: 104 - DNA, 28 bp, Homo sapiens
atgatgctggcctcataaaaagagttag

SEQ ID NO: 105 - DNA, 26 bp, Homo sapiens
tatcctagcttggcctgtttaagacc

SEQ ID NO: 106 - DNA, 22 bp, Homo sapiens
tgaggcaggagaatggcgtgaa

SEQ ID NO: 107 - DNA, 26 bp, Homo sapiens
tttaatatacctgctgatcaatgata

SEQ ID NO: 108 - DNA, 21 bp, Homo sapiens
gacacatgggatcatagcaaa

SEQ ID NO: 109 - DNA, 24 bp, Homo sapiens
ttgggcgacagagcaagacgactc

SEQ ID NO: 110 - DNA, 26 bp, Homo sapiens
atttggtcagtgggggctctgttaag

SEQ ID NO: 111 - DNA, 26 bp, Homo sapiens
tcagcagctgaaagaaatctgagtac

SEQ ID NO: 112 - DNA, 22 bp, Homo sapiens
gcgatacccaaagtcaatagtc

SEQ ID NO: 113 - DNA, 24 bp, Homo sapiens
gaagctgcagtaagccgagattgt

SEQ ID NO: 114 - DNA, 24 bp, Homo sapiens
gccctcttaactcccatgacattc

SEQ ID NO: 115 - DNA, 22 bp, Homo sapiens
agcctgggcgacagagcgagtc

SEQ ID NO: 116 - DNA, 23 bp, Homo sapiens
ctcggggctcgggagatgagtga

SEQ ID NO: 117 - DNA, 28 bp, Homo sapiens
cagcctaggtaacagagcaagacctttg

SEQ ID NO: 118 - DNA, 23 bp, Homo sapiens
gtttgcgtgatttgcgtggactt

SEQ ID NO: 119 - DNA, 23 bp, Homo sapiens
ctcctgcctcatcctcccgagta

SEQ ID NO: 120 - DNA, 24 bp, Homo sapiens
ccgagatcacgccactgcactcta

SEQ ID NO: 121 - DNA, 28 bp, Homo sapiens
tctcatttgagtggtggaagtgactggt

SEQ ID NO: 122 - DNA, 23 bp, Homo sapiens
tattctttcgggatgtaatctct

SEQ ID NO: 123 - DNA, 26 bp, Homo sapiens
cccgtctctactaaaaatactaaaac

SEQ ID NO: 124 - DNA, 28 bp, Homo sapiens
aaaccaacaataaggcaacctcttagtc

SEQ ID NO: 125 - DNA, 28 bp, Homo sapiens
tgccagagtagggtggtccatggtactt

SEQ ID NO: 126 - DNA, 25 bp, Homo sapiens
gcccaaaatgtgtttagttagcttc

SEQ ID NO: 127 - DNA, 28 bp, Homo sapiens
aggctgaagcaggagaatcacttaaaac

SEQ ID NO: 128 - DNA, 25 bp, Homo sapiens
gccaagtgtcgcttgtaattctatt

SEQ ID NO: 129 - DNA, 26 bp, Homo sapiens
gaatcttgtttcggcctttgacctta

SEQ ID NO: 130 - DNA, 25 bp, Homo sapiens
cgagatcacgccaccgcactctagc

SEQ ID NO: 131 - DNA, 27 bp, Homo sapiens
aaatctacccagctctgtaacgagaga

SEQ ID NO: 132 - DNA, 28 bp, Homo sapiens
aagctctgtttggcaagtgttaattgta

SEQ ID NO: 133 - DNA, 28 bp, Homo sapiens
ttggaatgtattctctgggtttggcagt

SEQ ID NO: 134 - DNA, 27 bp, Homo sapiens
ttcaggaggctgaggtgggaggattgt

SEQ ID NO: 135 - DNA, 22 bp, Homo sapiens
acctaggcaataccatctaaga

SEQ ID NO: 136 - DNA, 24 bp, Homo sapiens
gttgcctgttcactctgatagtct

SEQ ID NO: 137 - DNA, 23 bp, Homo sapiens
agcctgggtgacagagcgagact

SEQ ID NO: 138 - DNA, 28 bp, Homo sapiens
ttagagttatttgttgggatgagaatct

SEQ ID NO: 139 - DNA, 22 bp, Homo sapiens
ctgggcgacagagcgagactcc

SEQ ID NO: 140 - DNA, 26 bp, Homo sapiens
tctcctgccttagcctcccgagtagc

SEQ ID NO: 141 - DNA, 27 bp, Homo sapiens
tcctctccctaaaaagctccccctaag

SEQ ID NO: 142 - DNA, 28 bp, Homo sapiens
aggtcaaggctgcggtaagctgtgatcg

SEQ ID NO: 143 - DNA, 28 bp, Homo sapiens
tccccactttgtcctgcacactcctacc

SEQ ID NO: 144 - DNA, 23 bp, Homo sapiens
gggcgacagagcgagactccgtc

SEQ ID NO: 145 - DNA, 28 bp, Homo sapiens
aagatttaatagacatgcgcagaacact

SEQ ID NO: 146 - DNA, 23 bp, Homo sapiens
ccagcctgggcaaaagagcaagt

SEQ ID NO: 147 - DNA, 28 bp, Homo sapiens
acaaacatgaaaaggcaaatgatagaac

SEQ ID NO: 148 - DNA, 27 bp, Homo sapiens
agaggttgcagtgagccaagattgtag

SEQ ID NO: 149 - DNA, 23 bp, Homo sapiens
gagggatgaagggggacagatag

SEQ ID NO: 150 - DNA, 23 bp, Homo sapiens
cattctcactccacgccctctat

SEQ ID NO: 151 - DNA, 24 bp, Mus sp.
catagacccagtgctcatcttcgt

SEQ ID NO: 152 - DNA, 20 bp, Mus sp.
cattcggtggaaagctctga

SEQ ID NO: 153 - DNA, 20 bp, Mus sp.
tcaccatccattgcacagtt

SEQ ID NO: 154 - DNA, 20 bp, Mus sp.
ctgcgagaaggtactcaccc

SEQ ID NO: 155 - DNA, 22 bp, Mus sp.
atttggctttcaagcatccata

SEQ ID NO: 156 - DNA, 21 bp, Mus sp.
gggaagactgcttagggaaga

SEQ ID NO: 157 - DNA, 20 bp, Mus sp.
tctgcccaaacgtgcttaat

SEQ ID NO: 158 - DNA, 20 bp, Mus sp.
cctgcctgggctaaaataga

SEQ ID NO: 159 - DNA, 20 bp, Mus sp.
taccattcctaacagggttc

SEQ ID NO: 160 - DNA, 20 bp, Mus sp.
tttatgggtgtaatgcggtg

SEQ ID NO: 161 - DNA, 24 bp, Mus sp.
aattctattcatcgtctcggaagt

SEQ ID NO: 162 - DNA, 24 bp, Mus sp.
ttgagagattttatgggtgtaatg

SEQ ID NO: 163 - DNA, 22 bp, Mus sp.
tctctaggcctagcatatgaat

SEQ ID NO: 164 - DNA, 24 bp, Mus sp.
ttgaagaaggtagatggcatattg

SEQ ID NO: 165 - DNA, 24 bp, Mus sp.
caaaacccaatcacctaaggctaa

SEQ ID NO: 166 - DNA, 20 bp, Mus sp.
ttttggggtttggcattaag

SEQ ID NO: 167 - DNA, 23 bp, Mus sp.
attctgcgagaaggtactcaccc

SEQ ID NO: 168 - DNA, 27 bp, Mus sp.
attactagaatcgtacattgtccaaaa

SEQ ID NO: 169 - DNA, 26 bp, Homo sapiens
gtggtcttctacttgtgtcaatacag

SEQ ID NO: 170 - DNA, 23 bp, Homo sapiens
gaactcaagtccaaaaaatgagg

SEQ ID NO: 171 - DNA, 26 bp, Homo sapiens
atttatattttacacatttttgggcc

SEQ ID NO: 172 - DNA, 23 bp, Homo sapiens
tgacagtaaaatgaaaacattgc

SEQ ID NO: 173 - DNA, 22 bp, Homo sapiens
attagcatgggtgacagagcta

SEQ ID NO: 174 - DNA, 22 bp, Homo sapiens
ccaattacatagtcctcctttc

SEQ ID NO: 175 - DNA, 24 bp, Homo sapiens
ttcaatcatacacccatatctgtc

SEQ ID NO: 176 - DNA, 23 bp, Homo sapiens
attatagagggataggtaggcag

SEQ ID NO: 177 - DNA, 25 bp, Homo sapiens
ccaactctcatctgtattatctatg

SEQ ID NO: 178 - DNA, 27 bp, Homo sapiens
attttatccctgagtagcagaagaatg

SEQ ID NO: 179 - DNA, 21 bp, Homo sapiens
tcgagttgttatggttttagg

SEQ ID NO: 180 - DNA, 24 bp, Homo sapiens
atttggcttggaattcttttaccc

SEQ ID NO: 181 - DNA, 20 bp, Homo sapiens
tggggaatagttgaacggta

SEQ ID NO: 182 - DNA, 25 bp, Homo sapiens
attgcaacaagagtgaaactccatt

SEQ ID NO: 183 - DNA, 23 bp, Homo sapiens
attgactatgggcgtgagtgcat

SEQ ID NO: 184 - DNA, 22 bp, Homo sapiens
agaccctgtcattcacagatga

SEQ ID NO: 185 - DNA, 28 bp, Homo sapiens
actactgagtttctgttatagtgttttt

SEQ ID NO: 186 - DNA, 22 bp, Homo sapiens
gtcaatctctgcacctggaaat

SEQ ID NO: 187 - DNA, 23 bp, Homo sapiens
atttagaggcagtcatcgcagtg

SEQ ID NO: 188 - DNA, 22 bp, Homo sapiens
acctaccaatcccattccttag

SEQ ID NO: 189 - DNA, 24 bp, Homo sapiens
cggagtcgctggcacagttctatt

SEQ ID NO: 190 - DNA, 25 bp, Homo sapiens
tcgcgtttacaaacaagaaaagtgt

SEQ ID NO: 191 - DNA, 24 bp, Homo sapiens
tgactacttttgacttcagccagt

SEQ ID NO: 192 - DNA, 24 bp, Homo sapiens
aaccattcaacatttttaaccctt

SEQ ID NO: 193 - DNA, 20 bp, Homo sapiens
tcgcctccaagaatgtaagt

SEQ ID NO: 194 - DNA, 24 bp, Homo sapiens
atttctgcattttaactatggctc

SEQ ID NO: 195 - DNA, 20 bp, Homo sapiens
ccattgctgaattttacctc

SEQ ID NO: 196 - DNA, 20 bp, Homo sapiens
attgtgccattgcattccaa

SEQ ID NO: 197 - DNA, 24 bp, Homo sapiens
tgtgaaccacctatgaattgcaga

SEQ ID NO: 198 - DNA, 26 bp, Homo sapiens
attgcttgcagtgagcagagatcgtt

SEQ ID NO: 199 - DNA, 24 bp, Homo sapiens
catggcattggggacatgaacaca

SEQ ID NO: 200 - DNA, 24 bp, Homo sapiens
cactgagcgcttctagggacttct

SEQ ID NO: 201 - DNA, 24 bp, Homo sapiens
cagcctaggtgacagagcaagaca

SEQ ID NO: 202 - DNA, 24 bp, Homo sapiens
atttgcctaacctatggtcataac

SEQ ID NO: 203 - DNA, 28 bp, Homo sapiens
gaggctgaggcaggagaatggcgtgaac

SEQ ID NO: 204 - DNA, 28 bp, Homo sapiens
cgctgacgcagaacctgaaattgtgatt

SEQ ID NO: 205 - DNA, 26 bp, Homo sapiens
tatcctagcttggcctgtttaagacc

SEQ ID NO: 206 - DNA, 22 bp, Homo sapiens
tgaggcaggagaatggcgtgaa

SEQ ID NO: 207 - DNA, 28 bp, Homo sapiens
tctcatttgagtggtggaagtgactggt

SEQ ID NO: 208 - DNA, 23 bp, Homo sapiens
tattctttcgggatgtaatctct

SEQ ID NO: 209 - DNA, 28 bp, Homo sapiens
aggctgaagcaggagaatcacttaaaac

SEQ ID NO: 210 - DNA, 25 bp, Homo sapiens
gccaagtgtcgcttgtaattctatt

SEQ ID NO: 211 - DNA, 28 bp, Homo sapiens
ctaacttcccagcaacttcctttacact

SEQ ID NO: 212 - DNA, 24 bp, Homo sapiens
attgggcagacactgaactagctt

SEQ ID NO: 213 - DNA, 28 bp, Homo sapiens
cagcctaggtaacagagcaagacctttg

SEQ ID NO: 214 - DNA, 23 bp, Homo sapiens
gtttgcgtgatttgcgtggactt

SEQ ID NO: 215 - DNA, 26 bp, Homo sapiens
tcagcagctgaaagaaatctgagtac

SEQ ID NO: 216 - DNA, 22 bp, Homo sapiens
gcgatacccaaagtcaatagtc

SEQ ID NO: 217 - DNA, 24 bp, Homo sapiens
gaagctgcagtaagccgagattgt

SEQ ID NO: 218 - DNA, 24 bp, Homo sapiens
gccctcttaactcccatgacattc

SEQ ID NO: 219 - DNA, 20 bp, Homo sapiens
catttcttctgcccccatga

SEQ ID NO: 220 - DNA, 26 bp, Homo sapiens
atttgacagctgaaaaggtgcagatg

SEQ ID NO: 221 - DNA, 21 bp, Homo sapiens
gaggagacagccagggatata

SEQ ID NO: 222 - DNA, 27 bp, Homo sapiens
atttctctataacctctctccctatct

SEQ ID NO: 223 - DNA, 24 bp, Homo sapiens
ggaggaaaagtcttaaacgtgaat

SEQ ID NO: 224 - DNA, 24 bp, Homo sapiens
attggccttgatgtgtttgttact

SEQ ID NO: 225 - DNA, 20 bp, Homo sapiens
gccgatcctgaagcaatagc

SEQ ID NO: 226 - DNA, 23 bp, Homo sapiens
attccccttggtggtgtcatcct

SEQ ID NO: 227 - DNA, 20 bp, Homo sapiens
gtttgcatgtgaacaggtca

SEQ ID NO: 228 - DNA, 23 bp, Homo sapiens
attggcaggcaggtagatagaca

SEQ ID NO: 229 - DNA, 20 bp, Homo sapiens
ggctgcagggcataacatta

SEQ ID NO: 230 - DNA, 24 bp, Homo sapiens
attctatgactttgcgcttcagga

SEQ ID NO: 231 - DNA, 20 bp, Homo sapiens
gcacagaacaggcacttagg

SEQ ID NO: 232 - DNA, 18 bp, Homo sapiens
cgctcaaacgtgaggttg

SEQ ID NO: 233 - DNA, 32 bp, Homo sapiens
attgcaacttatatgtatttttgtatttcatg

SEQ ID NO: 234 - DNA, 28 bp, Homo sapiens
accaaattgtgttcatgagtatagtttc

SEQ ID NO: 235 - DNA, 33 bp, Homo sapiens
gccctagtggatgataagaataatcagtatgtg

SEQ ID NO: 236 - DNA, 30 bp, Homo sapiens
ggacagatgataaatacataggatggatgg

SEQ ID NO: 237 - DNA, 19 bp, Homo sapiens
ccctgggctctgtaaagaa

SEQ ID NO: 238 - DNA, 24 bp, Homo sapiens
atcagagcttaaactgggaagctg

SEQ ID NO: 239 - DNA, 26 bp, Homo sapiens
attaccaacatgaaagggtaccaata

SEQ ID NO: 240 - DNA, 33 bp, Homo sapiens
tgggttattaattgagaaaactccttacaattt

SEQ ID NO: 241 - DNA, 20 bp, Homo sapiens
ttcttgagcccagaaggtta

SEQ ID NO: 242 - DNA, 27 bp, Homo sapiens
attctaccagcaacaacacaaataaac

SEQ ID NO: 243 - DNA, 22 bp, Homo sapiens
atatgtgagtcaattccccaag

SEQ ID NO: 244 - DNA, 26 bp, Homo sapiens
tgtattagtcaatgttctccagagac

SEQ ID NO: 245 - DNA, 21 bp, Homo sapiens
gtgattcccattggcctgttc

SEQ ID NO: 246 - DNA, 22 bp, Homo sapiens
attcctgtgggctgaaaagctc

SEQ ID NO: 247 - DNA, 19 bp, Homo sapiens
actgcagtccaatctgggt

SEQ ID NO: 248 - DNA, 21 bp, Homo sapiens
atgaaatcaacagaggcttgc

SEQ ID NO: 249 - DNA, 19 bp, Homo sapiens
gaaggtcgaagctgaagtg

SEQ ID NO: 250 - DNA, 27 bp, Homo sapiens
attagaattctttaatctggacacaag

SEQ ID NO: 251 - DNA, 24 bp, Homo sapiens
ccggaggtaaaggtgtcttaaagt

SEQ ID NO: 252 - DNA, 22 bp, Homo sapiens
atttcctgtgtcagaccctgtt

SEQ ID NO: 253 - DNA, 24 bp, Homo sapiens
gggggtctaagagcttgtaaaaag

SEQ ID NO: 254 - DNA, 29 bp, Homo sapiens
gtttgtgtgtgcatctgtaagcatgtatc

SEQ ID NO: 255 - DNA, 21 bp, Homo sapiens
atgttggtcaggctgactatg

SEQ ID NO: 256 - DNA, 24 bp, Homo sapiens
gattccacatttatcctcattgac

SEQ ID NO: 257 - DNA, 26 bp, Homo sapiens
attacagaagtctgggatgtggagga

SEQ ID NO: 258 - DNA, 19 bp, Homo sapiens
ggcagcccaaaaagacaga

SEQ ID NO: 259 - DNA, 23 bp, Homo sapiens
ggtgattttcctctttggtatcc

SEQ ID NO: 260 - DNA, 26 bp, Homo sapiens
agccacagtttacaacatttgtatct